(19) United States
(12) Patent Application Publication
(10) Pub. No.: US 2024/0110139 A1
(43) Pub. Date: Apr. 4, 2024

(54) CELL CULTURE SYSTEMS, METHODS AND USES THEREOF

(71) Applicant: ACADEMIA SINICA, TAIPEI (TW)

(72) Inventor: YING-CHIH CHANG, ATHERTON, CA (US)

(73) Assignee: ACADEMIA SINICA, TAIPEI (TW)

(21) Appl. No.: 18/267,996

(22) PCT Filed: Dec. 30, 2021
(86) PCT No.: PCT/US2021/065694

§ 371 Date: Jun. 16, 2023

Related U.S. Application Data
(60) Provisional application No. 63/132,934, filed on Dec. 31, 2020.
(60) Provisional application No. 63/252,268, filed on Oct. 5, 2021.

(51) Int. Cl. C12M 1/00; C12M 1/32

(52) U.S. Cl. CPC C12M 23/20; C12M 23/12; C12N 2513/00; C12N 2533/40; C12N 2537/10

(57) ABSTRACT

A surface coating comprising a hydrophilic polymer and polyelectrolyte multilayers. Also, a cell culture system including a cell culture article having a surface coated with the surface coating. Also, methods of preparing the surface coatings and systems.

### CROSS-REFERENCE TO RELATED APPLICATIONS

This application claims priority to and the benefit of U.S. Provisional Patent Application No. 63/132,934, filed Dec. 31, 2020 and U.S. Provisional Patent Application No. 63/252,268, filed Oct. 5, 2021, the disclosure of which is hereby incorporated herein by reference in its entirety.

### BACKGROUND OF THE INVENTION

The interest in 3D spheroid models is growing among researchers, from basic science to preclinical drug discovery applications, including studies in tumor biology, neurodegenerative diseases, and drug toxicity. Three-dimensional (3D) cell culture methods are increasingly used to generate complex tissue or tumor models.

There is a lot of variation in the spheroids formed using 3D cell culture methods and products available on the market, and this may impact their read-out. For instance, the widely used non-adherent techniques for 3D cell culture, including Ultra Low Attachment (ULA) plate and hanging drop method, have not proven suitable because these methods usually generate spheroids via cell agglomeration. Such spheroids generally maintain their original heterogeneity and harbor multiple cells with various characteristics, requiring a better understanding of cellular heterogeneity. When tens-of-thousands cells are aggregated into a spheroid (i.e., a mass with spherical shape), an extensive central necrotic core may form over a few hours due to the lack of nutrient and oxygen penetration, and thus hinders cell proliferation. Extended central necrosis is a rare phenomenon in real cancers.

Alternatively, Matrigel is a commonly used embedded substrate for tissue-based cell growth, such as organoid formation. But out of focus, inefficient compound diffusion, and difficulty in sample isolation limits its application for ex vivo 3D spheroid-based applications.

Standardizing spheroid formation is critical to generating uniform 3D cell culture and obtaining reproducible results from spheroid-based assays and drug screening. Therefore, there is a need for the development of new cell culture systems and methods that can reliably form single cell-derived spheroids.

### SUMMARY OF THE INVENTION

The present disclosure provides a surface coating for coating a cell culture article. The surface coating described herein comprises a hydrophilic polymer and polyelectrolyte multilayers. The substrate provided herein is advantageous for hydration preservation. It can prevent the cell culture substrate from undesirable surface cracks caused by prolonged storage at ambient temperature. In some embodiments, the surface coating provided herein enables the formation of single-cell derived spheroids derived from single cells. Also provided is a cell culture system comprising the cell culture article. Uses and methods of preparing the surface coatings and systems are provided as well.

Accordingly, one aspect of the present disclosure provides a composition for coating a surface of a cell culture article. The composition described herein comprises a) a hydrophilic polymer, in which the hydrophilic polymer is deposited on a surface of the cell culture article, and b) polyelectrolyte multilayers, in which the hydrophilic polymer is in direct contact with a polycation or an polyanion of the polyelectrolyte multilayers.

The cell culture article described herein may be made of any suitable plastics or polymers such as polyethylene, polypropylene, polymethylpentene, cyclic olefin polymer, cyclic olefin copolymer, polyvinyl chloride, polyurethane, polyester, polyamide, ethylene-vinyl acetate copolymer, ethylene-vinyl alcohol copolymer, ethylene-acrylic acid copolymer, ethylene-methyl acrylate copolymer, ethylene-methacrylic acid copolymer, ethylene-methyl methacrylate copolymer, polyacrylic acid, polymethacrylic acid, methyl polyacrylate, and methyl polymethacrylate, or derivatives of these or the like.

The surface coating described herein may be dehydrated or hydrated. In some embodiments, the surface coating is in a dehydrated state. In some embodiments, the surface coating is in a hydrated state.

Suitable hydrophilic polymers include, but are not limited to, poly(vinyl alcohol) (PVA), poly(ethylene glycol) (PEG), PEG-acrylate, polyvinylpyrrolidone (PVP), polyethyleneimine (PEI), poly-L-lactide (PLLA), poly-D-lactide (PDLA), poly(L-lactide-co-D,L-lactide) (PLDLLA), poly(glycolic acid) (PGA), poly(lactic-co-glycolic acid) (PL-co-GA), poly(methyl methacrylate) (PMMA), poly(hydroxyethyl methacrylate) (p-HEMA) and derivatives thereof.

In some embodiments, the hydrophilic polymer is PVA, PEG, PVP, PEI, PMMA or a derivative thereof. In some embodiments, the absorbent polymer is PVA. In some embodiments, the hydrophilic polymer is PEG or PEG-acrylate such as PEGMA, PEGDMA or PEGDA. In some embodiments, the hydrophilic polymer is PLA or a derivative such as PLLA, PDLA or PLDLLA. In some embodiments, the hydrophilic polymer is PGA or a derivative such as PLGA. In some embodiments, the hydrophilic polymer is PMAA or a derivative such as pHEMA.

In certain embodiments, the volume of the hydrophilic polymer (e.g. PVA) is 0.01-10% of the total volume of the surface coating.

The polyelectrolyte multiplayers described herein comprise at least one layer pair (referred as “bilayer”) comprising a cationic polyelectrolyte (referred as “polycation”) and an polyelectrolyte (referred as “polyanion”). In some embodiments, the polycation is a poly(amino acid). In some embodiments, the polyanion is a poly(amino acid). In some embodiments, the polycation and the polyanion are poly(amino acid)s. The poly(amino acid)s described herein may comprise L and/or D amino-acid forms. As described herein, the polyelectrolyte multiplayers can be formed by depositing polycations and polyanions in an alternative fashion via layer-by-layer assembly.

In some embodiments, the polyelectrolyte multilayers having a formula of (polycation/polyanion)ₙ comprise n bilayers of polycations and polyanions, wherein n is an integer number ranging from 1 to 30. In some embodiments, n is 1, 2, 3, 4, 5, 6, 7, 8, 9, 10, 11, 12, 13, 14, 15, 16, 17, 18, 19 or 20. In some embodiments, n is in a range of 1-10, 1-8, 1-5, 3-20, 5-20, 10-20, 11-19, 12-18, 13-17, or 14-16.

In some embodiments, the polyelectrolyte multilayers having a formula of polyanion(polycation/polyanion)ₙ comprise n+1 layers of polyanions and n layers of polycations, wherein n is an integer number ranging from 1 to 30. In some embodiments, n is 1, 2, 3, 4, 5, 6, 7, 8, 9, 10, 11, 12, 13, 14, 15, 16, 17, 18, 19 or 20. In some embodiments, n is in a range of 1-10, 1-8, 1-5, 3-20, 5-20, 10-20, 11-19, 12-18, 13-17, or 14-16.

In some embodiments, the polyelectrolyte multilayers having a formula of polycation(polyanion/polycation)ₙ comprise n+1 layers of polycations and n layers of polyanions, wherein n is an integer number ranging from 1 to 30. In some embodiments, n is 1, 2, 3, 4, 5, 6, 7, 8, 9, 10, 11, 12, 13, 14, 15, 16, 17, 18, 19 or 20. In some embodiments, n is in a range of 1-10, 1-8, 1-5, 3-20, 5-20, 10-20, 11-19, 12-18, 13-17, or 14-16.

In some embodiments, the polycation is poly(L-lysine) (PLL), poly(L-arginine) (PLA), poly(L-ornithine) (PLO), poly(L-histidine) (PLH), or a combination thereof. In a preferred embodiment, the polycation is PLL.

In preferred embodiments, the polyanion is poly(L-glutamic acid) (PLGA), poly(L-aspartic acid) (PLAA), or a combination thereof. In a preferred embodiment, the polyanion is PLGA.

In some embodiments, said polyelectrolyte multilayers comprise at least one layer pair (i.e., bilayer) of polycation/polycation selecting from the group consisting of PLL/PLGA, PLL/PLAA, PLA/PLGA, PLA/PLAA, PLO/PLGA, PLO/PLAA, PLH/PLGA, PLH/PLAA, and a combination thereof.

In some embodiments, the bilayer described herein comprises a combination of PLL and PLGA. In some embodiments, the bilayer described herein comprises a combination of PLO and PLGA. In some embodiments, the bilayer described herein comprises a combination of PLH and PLGA. In some embodiments, the bilayer described herein comprises a combination of PLA and PLGA.

In some embodiments, the bilayer described herein comprises a combination of PLL and PLAA. In some embodiments, the bilayer described herein comprises a combination of PLO and PLAA. In some embodiments, the bilayer described herein comprises a combination of PLH and PLAA. In some embodiments, the bilayer described herein comprises a combination of PLA and PLAA.

In some embodiments, the polyelectrolyte multilayers described herein may have a thickness ranging from 30 nm to 30 μm. In some embodiments, the surface coating has a thickness ranging from 100 nm to 20 μm. In some embodiments, the surface coating has a thickness of 200, 400, 600 or 800 nm. In some embodiments, the surface coating has a thickness of 1, 5, 10, 15 or 20 μm.

Compared with conventional culture methods, the surface coating of the present disclosure offers an improved proliferation rate for a variety of cells including, but not limited to, tumor cells, pluripotent and multipotent stem and progenitor cells, hematopoietic cells and immune cells. In addition, the surface coating with elevated water retention offers an advantage to prevent the surface coating from undesirable surface cracks caused by dehydration due to prolonged storage at ambient temperature.

In another aspect, the present invention provides methods for coating a cell culture article. The method described herein comprises the steps of: (a) providing a cell culture article having a hydrophobic surface; (b) modifying the hydrophobic surface with a treatment; (c) applying a hydrophilic polymer to the modified surface; and (d) sequentially depositing on the hydrophilic polymer alternating layers of polycations and polyanions.

In some embodiments, the treatment described herein is a plasma treatment, corona discharge or UV ozone treatment. In some embodiments, the hydrophobic surface described herein is irradiated or hydrophilized after the treatment. In some embodiments, the hydrophobic surface is hydrophilized after applying the hydrophilic polymer (e.g., PVA) to the surface. In some embodiments, the hydrophilic polymer (e.g., PVA) is covalently linked (i.e., conjugated) to the surface. A cross-linking agent may be used to facilitate the crosslinking (i.e., conjugation). Exemplary cross-linking agents include, but are not limited to, maleic acid, formaldehyde, glutaraldehyde, butanal (butyraldehyde), sodium borate, or a combination thereof.

In another aspect, the present invention provides a cell culture system comprising a cell culture article having a substrate with the inventive surface coating configured to culture cells. In some embodiments, the cell culture system further comprises cells. In some embodiments, the cells are adapted to be human cells. In some embodiments, the cells are adapted to be living cells. In some embodiments, the cell culture system further comprises culture media.

In some embodiments, the cell culture system disclosed herein enables an efficient and scalable multiplication of cells, in particular, single cells or low-density cells (e.g., cells with an abundance of less than 1000 in one milliliter) into 3D, making it possible to form 3D cell culture on difficult cell types that did not form on current platforms in the market (e.g., Ultra Low Attachment (ULA) plate, Hanging-Drop).

As disclosed herein, one or more parameters of the polyelectrolyte multilayers and the culture medium may be selected by the user, based on one or more microenvironment selection criteria for the cells.

The cell culture system disclosed herein enables not only cell attachment and growth, but also the viable harvest of cultured cells (e.g. 3D cell culture, tissue and organs). The inability to harvest viable cells is a significant drawback in current platforms on the market, and it leads to difficulty in building and sustaining a sufficient number of cells for production capacity. According to an aspect of embodiments of this disclosure, it is possible to harvest viable cells from the cell culture system, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. In some embodiments, cells can be released from the surface coating with using a cell dissociation enzyme, for example, trypsin, TrypLE, or Accutase. In preferred embodiments, cells can be released from the surface coating without using a cell dissociation enzyme.

In another aspect, the present invention provides methods for culturing cells using the cell culture article disclosed herein. The method for culturing cells comprises the steps of: a) providing a cell culture article having a surface coated with the surface coating of the present disclosure; b) seeding cells on the coated surface; c) culturing the cells under a suitable medium for a sufficient period of time to form one or more spheroids. In some embodiments, the spheroids generated herein are adhered to the substrate. In some embodiments, the spheroids generated herein are semi-attached to the substrate. In some embodiments, the spheroids are derived from single cells via single cell proliferation. The cultured cells (e.g., cultured and harvested cells) may be used for various applications such as analysis and characterization, screening drugs, isolating single-cell derived clone, generating cell banks, and generating animal models.

As described herein, the cells are living cells. In some embodiments, the cells are mammalian cells. In some embodiments, the cells are tissue cells, immune cells, endothelial cells, stem cells, epithelial cells, mesenchymal cells, mesothelial cells, tumor cells or tumor-associated cells.

As described herein, culturing the cells comprise maintaining and/or proliferating cells. In some embodiments, culturing the cells comprises maintaining cells. In some embodiments, culturing the cells comprises proliferating cells. In some embodiments, culturing the cells may further comprise differentiating cells.

In some embodiments, the cells are stem cells such as mesenchymal stem cells (MSCs) or pluripotent stem cells (PSCs) including embryonic stem cells (ESCs) and induced pluripotent stem cells (iPSCs).

In some embodiments, the cells are tumor cells, and the cultured cells are tumor spheroids. The tumor spheroids may be derived from a cell line, a tumor tissue or a liquid biopsy. In some embodiments, tumor spheroids described herein are derived from circulating tumor cells (CTCs) isolated from a blood sample obtained from a cancer patient. In some embodiments, the blood sample described herein is a whole blood. The blood sample can be obtained by liquid biopsy. In some embodiments, the cancer patient described herein is a human cancer patient having a metastatic cancer. In some embodiments, the blood sample is obtained from the cancer patient before, during, and/or after therapeutic treatment.

Another aspect of the present disclosure provides a method of preparing a single-cell derived spheroid in vitro, the method comprising the steps of: (a) providing a cell culture system comprising the substrate of the present disclosure; (b) isolating cells (e.g. tumor cells and/or tumor-associated cells) from a sample to provide isolated cells; (c) seeding the isolated cells on the substrate; and (d) culturing the cells under a suitable medium for a time sufficient to produce one or more spheroids, wherein the one or more spheroids are single-cell derived.

Another aspect of the present disclosure provides methods for isolating single cell derived clones, each composed of a homogenous cell population that is genetically identical.

### BRIEF DESCRIPTION OF THE DRAWINGS

FIG. 1A is a side cross-sectional view of an embodiment of the surface coating of the present disclosure. A hydrophilic polymer 102 is deposited on a surface of the well 101 in the cell culture article 202. 102 and the surface of the well 101 are not crosslinked. 301 is a polyanion. 302 is a polycation. 103 is an embodiment of polyelectrolyte multiplayers including 4 bilayers of 301 and 302. The outermost layer is 301. 102 is in direct contact with 302.

FIG. 1B is a side cross-sectional view of an embodiment of the surface coating of the present disclosure. A hydrophilic polymer 102 is deposited on a surface of the well 101 in the cell culture article 202. 102 and the surface of the well 101 are crosslinked. 301 is a polyanion. 302 is a polycation. 103 is an embodiment of polyelectrolyte multiplayers including 4 bilayers of 301 and 302. The outermost layer is 301. 102 is in direct contact with 302.

FIG. 2A is a side cross-sectional view of an embodiment of the surface coating of the present disclosure. A hydrophilic polymer 102 is deposited on a surface of the well 101 in the cell culture article 202. 102 and the surface of the well 101 are not crosslinked. 301 is a polyanion. 302 is a polycation. 103 is an embodiment of polyelectrolyte multiplayers including 5 layers of 302 and 4 layers of 301. The outermost layer is 302. 102 is in direct contact with 302.

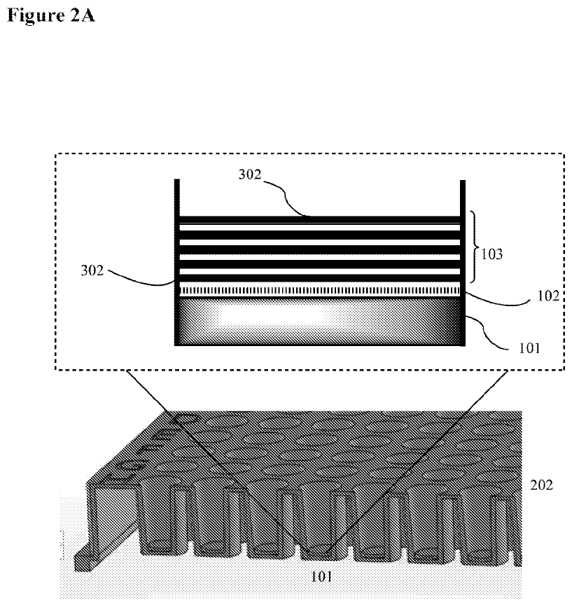

FIG. 2B is a side cross-sectional view of an embodiment of the surface coating of the present disclosure. A hydrophilic polymer 102 is deposited on a surface of the well 101 in the cell culture article 202. 102 and the surface of the well 101 are crosslinked. 301 is a polyanion. 302 is a polycation. 103 is an embodiment of polyelectrolyte multiplayers including 5 layers of 302 and 4 layers of 301. The outermost layer is 302. 102 is in direct contact with 302.

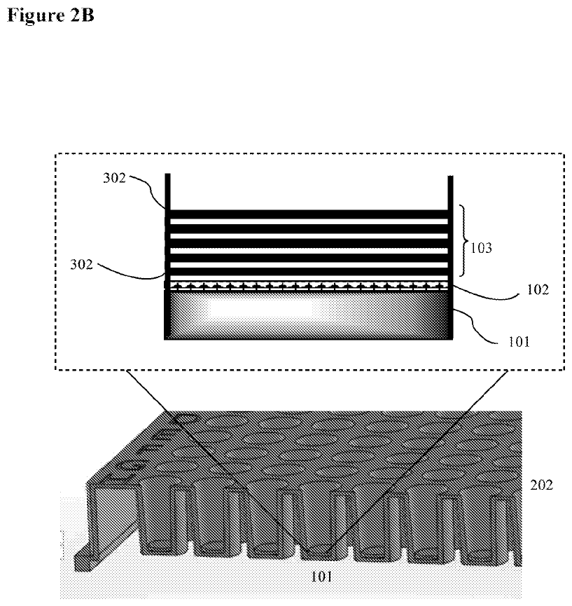

FIG. 3A is a side cross-sectional view of an embodiment of the surface coating of the present disclosure. A hydrophilic polymer 102 is deposited on a surface of the well 101 in the cell culture article 202. 102 and the surface of the well 101 are not crosslinked. 301 is a polyanion. 302 is a polycation. 103 is an embodiment of polyelectrolyte multiplayers including 4 bilayers of 301 and 302. The outermost layer is 302. 102 is in direct contact with 301.

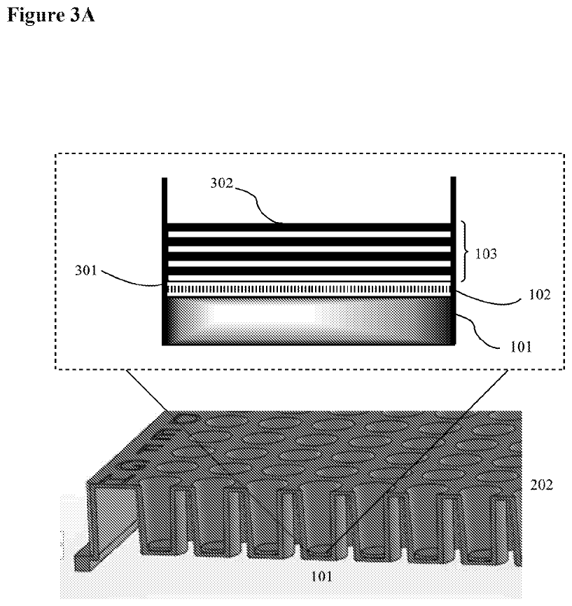

FIG. 3B is a side cross-sectional view of an embodiment of the surface coating of the present disclosure. A hydrophilic polymer 102 is deposited on a surface of the well 101 in the cell culture article 202. 102 and the surface of the well 101 are crosslinked. 301 is a polyanion. 302 is a polycation. 103 is an embodiment of polyelectrolyte multiplayers including 4 bilayers of 301 and 302. The outermost layer is 302. 102 is in direct contact with 301.

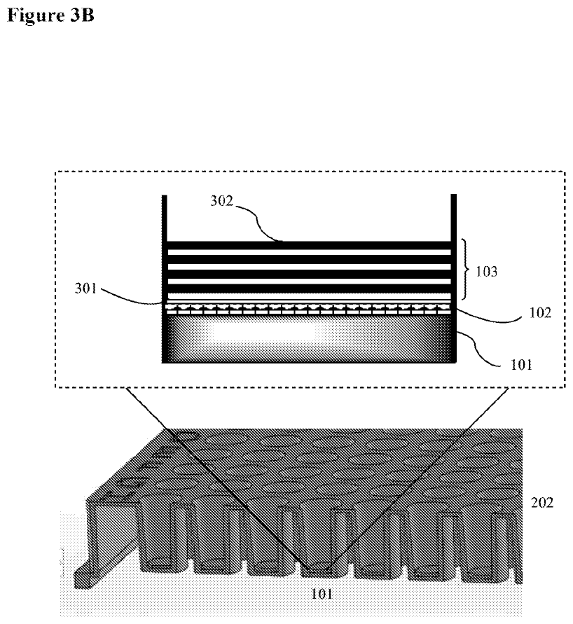

FIG. 4A is a side cross-sectional view of an embodiment of the surface coating of the present disclosure. A hydrophilic polymer 102 is deposited on a surface of the well 101 in the cell culture article 202. 102 and the surface of the well 101 are not crosslinked. 301 is a polyanion. 302 is a polycation. 103 is an embodiment of polyelectrolyte multiplayers including 5 layers of 301 and 4 layers of 302. The outermost layer is 301. 102 is in direct contact with 301.

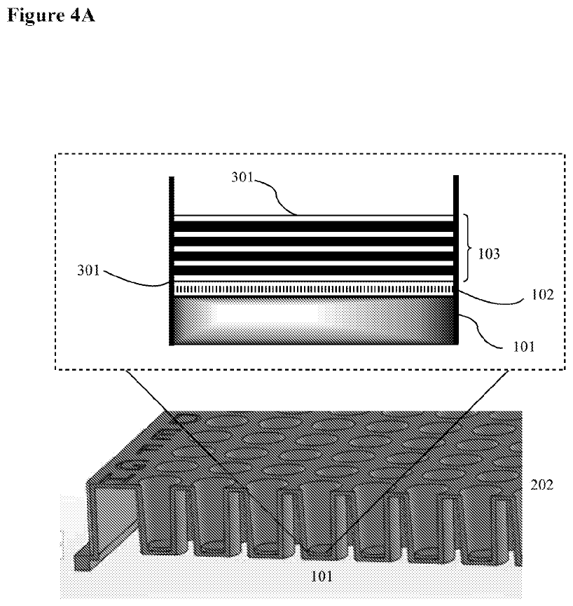

FIG. 4B is a side cross-sectional view of an embodiment of the surface coating of the present disclosure. A hydrophilic polymer 102 is deposited on a surface of the well 101 in the cell culture article 202. 102 and the surface of the well 101 are crosslinked. 301 is a polyanion. 302 is a polycation. 103 is an embodiment of polyelectrolyte multiplayers including 5 layers of 301 and 4 layers of 302. The outermost layer is 301. 102 is in direct contact with 301.

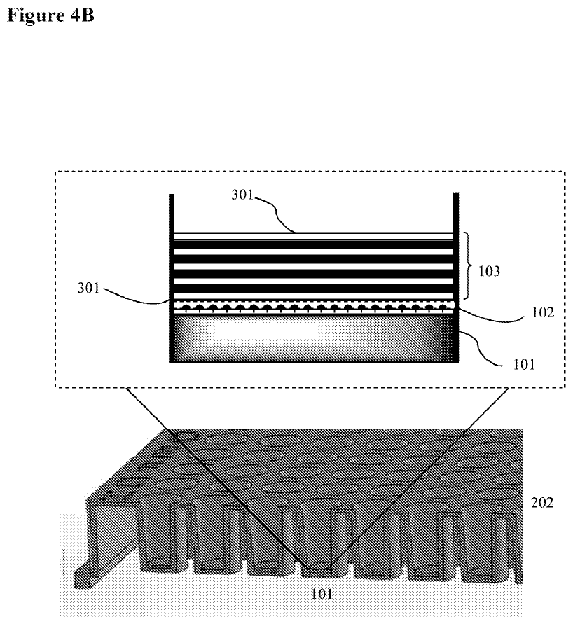

FIG. 5 illustrates of an embodiment of the surface modification of a cell culture article. A tissue culture plate made of polystyrene plastic is first treated by an ozone plasma, followed by addition of a photo-activated azidophenyl-PVA to the modified surface to form a PVA-crosslinked polystyrene plate.

FIG. 6 illustrates of an embodiment of the surface modification of a cell culture article. A tissue culture plate made of polytetrafluoroethylene (PTFE) is first treated by plasma gas, followed by depositing PVA onto the modified surface of the PTFE plate. A cross-linking agent, glutaraldehyde (GA), is applied to crosslink PVA to PTFE to form a PVA-crosslinked PTFE plate.

FIG. 7 shows the time-lapse microscope observation of HCT116 colorectal cancer cells cultured on the surface coating of the invention on day 0, 1, 2, 3, 4 and 5 during the growth of the cancer cells supplied with complete DMEM medium. (Image photographed by Leica DMI6000B time-lapse microscope under 10× objective).

FIGS. 8A-E show the results of ex vivo cultivation using the culture platform of the invention, and the formation of spheroids (after 7-14 days) derived from (A) lung cancer cell lines A549, H1299, PC-9 and H1975; (B) liver cancer cell lines SNU-398, SNU-475, PLC/PRF/S, Hep3B and Huh7 (C) breast cancer cell lines MDA-MB-231 and CGBC01; (D) colorectal cancer cell lines HCT116, HCT15 and WiDr; and (E) human tongue squamous carcinoma cell line SAS, ovarian cancer cell line SK-OV-3, and cell line T24 derived from a human urinary bladder cancer patient

FIGS. 9A-C show the representative time-dependent images of CTC-derived spheroid cultivation on the culture platform of the invention. (A) CTCs were isolated from a blood sample of a breast cancer patient; CTC-derived spheroids formed after 14 days. (B) CTCs were isolated from a blood sample of a head&neck cancer patient; CTC-derived spheroids formed after 38 days. (C) CTCs were isolated from a blood sample of a colorectal cancer patient; CTC-derived spheroids formed after 13-27 days. Scale bar: 50 μm.

FIGS. 10A-B show the images of tumor spheroids derived from primary colorectal tumor tissues obtained from colorectal cancer (CRC) patient. Tumor spheroids were generated on the culture platform of the invention after (A) 2 weeks and (B) 4 weeks.

### DETAILED DESCRIPTION OF THE INVENTION

The present disclosure relates to a new generation of scaffold-free 3D cell culture technology and uses thereof. In some embodiments, provided is a new composition for surface coating. Also provided is a cell culture system comprising a surface coating that is useful for cell culture, in particular, 3D cell culture. The surface coating can induce the formation of highly uniform 3D cell culture, making it possible to form 3D cell culture on difficult primary cell types that did not form on any other low attachment surface. Compared with conventional culture methods, the surface coating described herein offers an improved proliferation rate for a variety of cells including, but not limited to, tumor cells, pluripotent and multipotent stem and progenitor cells, hematopoietic cells and immune cells. In certain embodiments, the surface coating comprises a hydrophilic polymer (e.g. PVA), and one or more pairs of polyelectrolytes.

### Surface Coatings

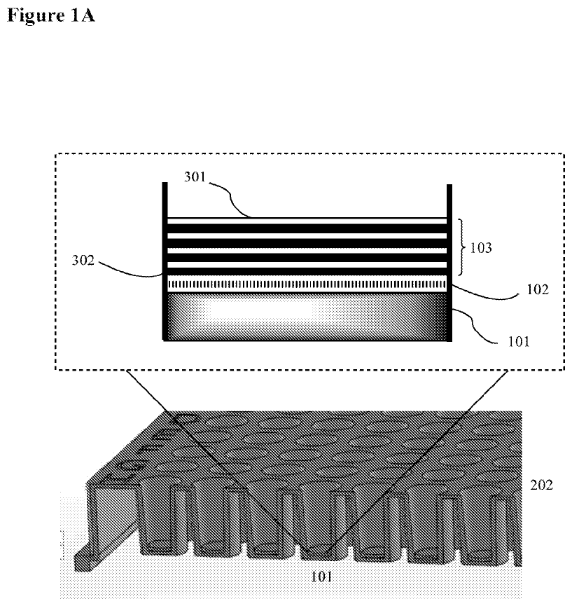

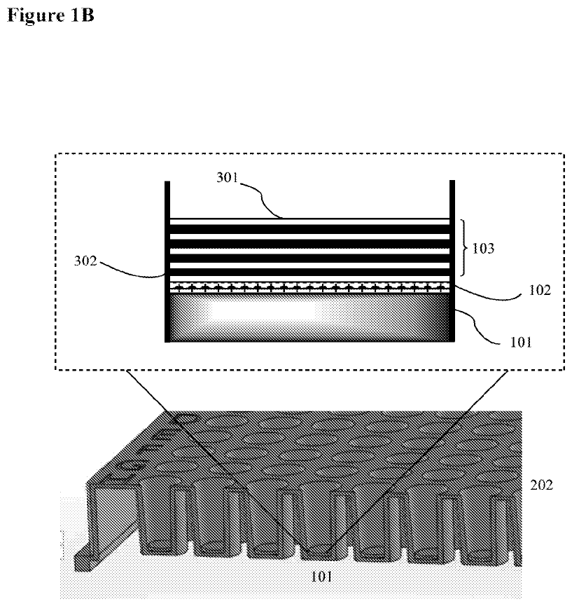

The surface coating of the present disclosure comprises a hydrophilic polymer and polyelectrolyte multilayers. In some instances, the surface coating is as illustrated in FIG. 1. As show in FIG. 1, 104 indicates an illustrative surface coating. The hydrophilic polymer 102 is deposited on the top surface of the well 201 of a cell culturing plate 202. Polyelectrolyte multilayers 103 is deposited on top of the hydrophilic polymer layer 102.

Without being bound to any particular theory, it is believed that the surface coating enables robust multiplication or stable maintenance of cells (e.g., rare cells extracted from blood, low-density cells, or single cells) seeded on the surface coating with or without the substrate for an extended period, for example, over 48 hours, over 72 hours, over 96 hours, over 5 days, over 6 days, over 7 days, or in one to several weeks (e.g., 1, 2, 3, 4, 5, 6, or more weeks).

1) Hydrophilic Polymers

Hydrophilic polymers described herein are hydrophilic absorbent polymers (“absorbent polymers”) that water soluble and may swell as a result of uptake and retention of aqueous solutions. A non-limiting list of hydrophilic absorbent polymers that may be used with the present invention includes hydrophilic and biocompatible grades of the following polymers and their derivatives: poly(vinyl alcohol) (PVA), ethylene vinyl alcohol co-polymers (typically non-biodegradable materials which degree of hydrophilicity depends on distribution of ethylene (hydrophobic) and vinyl alcohol (hydrophilic) groups), co-polymers of polyvinyl alcohol and ethylene vinyl alcohol, polyacrylate compositions, polyurethane compositions, poly(ethylene glycol) (PEG), otherwise known as poly(oxyethylene) (POE) and poly(ethylene oxide) (PEO), and its derivatives including but not limited to polyethylene glycol methacrylate (PEGMA), polyethylene glycol dimethacrylate (PEGDMA) and polyethylene glycol diacrylate (PEGDA); nitrogen-containing materials such as polyacrylamide (without acrylamide toxic residuals), polyvinylpyrrolidone, polyvinylamine, and polyethyleneimine; electrically charged materials such as poly(lactic acid) also known as polylactide in various forms (e.g. poly-L-lactide (PLLA) and its derivatives, poly-D-lactide (PDLA) and its derivatives, poly(L-lactide-co-D,L-lactide) (PLDLLA) and its derivatives), poly(glycolic acid) (PGA) also known as polyglycolide, co-polymers of lactic acid and glycolic acid poly(lactic-co-glycolic acid) (PL-co-GA), co-polymers of PLA and/or PGA with PEG; polymethacrylic acid; poly(hydroxyethyl methacrylate) (poly-HEMA), among other absorbent, hydrophilic and biocompatible materials known in the art.

In some embodiments, the hydrophilic absorbent polymer is selected from the group consisting of poly(vinyl alcohol) (PVA), copolymers of ethylene vinyl alcohol, copolymers of polyvinyl alcohol and ethylene vinyl alcohol, polyacrylate compositions, polyurethane compositions, poly(ethylene glycol) (PEG), PEG-acrylate, polyethylene glycol methacrylate (PEGMA), polyethylene glycol dimethacrylate (PEGDMA), polyethylene glycol diacrylate (PEGDA), polyacrylamide (PAM), polyvinylpyrrolidone (PVP), polyvinylamine (PVAm), polyethyleneimine (PEI), poly-L-lactide (PLLA), poly-D-lactide (PDLA), poly(L-lactide-co-D,L-lactide) (PLDLLA), poly(glycolic acid) (PGA), poly(lactic-co-glycolic acid) (PL-co-GA), poly(methyl methacrylate) (PMMA) and poly(hydroxyethyl methacrylate) (p-HEMA).

In some embodiments, the hydrophilic absorbent polymer is selected from the group consisting of PVA, PEG, PEG-acrylate, polylactide, PMMA, p-HEMA, a combination or a derivative thereof. In some embodiments, the absorbent polymer is PVA or a derivative thereof. In some embodiments, the absorbent polymer is PEG or PEG-acrylate such as PEGMA, PEGDMA or PEGDA. In some embodiments, the absorbent polymer is polylactide or a derivative such as PLLA, PDLA or PLDLLA. In some embodiments, the absorbent polymer is PGA or a derivative such as PLGA. In some embodiments, the absorbent polymer is PMAA or a derivative such as pHEMA.

In some embodiments, the hydrophilic polymer has an average molecular weight of from about 2,500 g/mol to about 200,000 g/mol. In some cases, the average molecular weight of the hydrophilic polymer is from about 5,000 g/mol to about 175,000 g/mol, from about 5,000 g/mol to about 150,000 g/mol, from about 5,000 g/mol to about 125,000 g/mol, from about 5,000 g/mol to about 100,000 g/mol, from about 5,000 g/mol to about 75,000 g/mol, from about 5,000 g/mol to about 50,000 g/mol, from about 5,000 g/mol to about 25,000 g/mol, from about 5,000 g/mol to about 10,000 g/mol, from about 10,000 g/mol to about 175,000 g/mol, from about 10,000 g/mol to about 150,000 g/mol, from about 10,000 g/mol to about 125,000 g/mol, from about 10,000 g/mol to about 100,000 g/mol, from about 10,000 g/mol to about 75,000 g/mol, from about 10,000 g/mol to about 50,000 g/mol, from about 10,000 g/mol to about 25,000 g/mol, from about 20,000 g/mol to about 150,000 g/mol, or from about 50,000 g/mol to about 150,000 g/mol.

In some instances, the hydrophilic polymer is deposited directly onto the surface of a target substrate. In other instances, the hydrophilic polymer is deposited indirectly onto the surface. In some cases, one or more additional layers (e.g., 1, 2, 3, 4, 5, or more layers) are formed between the hydrophilic polymer layer and the surface of the substrate. In some cases, one additional layer (also referred to herein as the innermost layer) is formed between the hydrophilic polymer layer and the surface of the substrate.

In some embodiments, the hydrophilic polymer is PVA. PVA can have an average molecular weight ranging from about 10,000 g/mol to about 125,000 g/mol. In some instances, PVA has an average molecular weight of from about 10,000 g/mol to about 100,000 g/mol, from about 10,000 g/mol to about 75,000 g/mol, from about 10,000 g/mol to about 50,000 g/mol, from about 20,000 g/mol to about 125,000 g/mol, from about 20,000 g/mol to about 100,000 g/mol, from about 20,000 g/mol to about 75,000 g/mol, from about 20,000 g/mol to about 50,000 g/mol, from about 50,000 g/mol to about 125,000 g/mol, or from about 50,000 g/mol to about 100,000 g/mol.

In some instances, the PVA is deposited directly onto the surface of a target substrate. In other instances, the PVA is deposited indirectly onto the surface. In some cases, one or more additional layers (e.g., 1, 2, 3, 4, 5, or more layers) are formed between the PVA layer and the surface. In some cases, one additional layer is formed between the PVA layer and the surface of the substrate.

In some embodiment, the hydrophilic polymer is PEG. In some instances, the average molecular weight of PEG is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some instances, the PEG utilized herein is a discrete PEG (dPEG). A discrete PEG can be a polymeric PEG comprising more than one repeating ethylene oxide units. In some cases, the discrete PEG comprises from 2 to 60, from 2 to 50, or from 2 to 48 repeating ethylene oxide units. In some cases, the dPEG comprises 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units.

In certain embodiments, the volume of the hydrophilic polymer (e.g. PVA or PEG) is from about 0.01% to about 10% of the total volume of the surface coating. In some instances, the hydrophilic polymer is from about 0.01% to about 9% v/v, from about 0.01% to about 8% v/v, from about 0.01% to about 7% v/v, from about 0.01% to about 6% v/v, from about 0.01% to about 5% v/v, from about 0.01% to about 4% v/v, from about 0.01% to about 3% v/v, from about 0.01% to about 2% v/v, from about 0.01% to about 1% v/v, from about 0.1% to about 10% v/v, from about 0.1% to about 9% v/v, from about 0.1% to about 8% v/v, from about 0.1% to about 7% v/v, from about 0.1% to about 6% v/v, from about 0.1% to about 5% v/v, from about 0.1% to about 4% v/v, from about 0.1% to about 3% v/v, from about 1% to about 10% v/v, from about 1% to about 9% v/v, from about 1% to about 8% v/v, from about 1% to about 7% v/v, from about 1% to about 6% v/v, from about 1% to about 5% v/v, from about 1% to about 4% v/v, from about 2% to about 10% v/v, or from about 5% to about 10% v/v, of the total volume of the surface coating. In some cases, the volume of the hydrophilic polymer (e.g. PVA or PEG) is about 0.01%, about 0.05%, about 0.1%, about 0.5%, about 1%, about 2%, about 3%, about 4%, about 5%, about 6%, about 7%, about 8%, about 9%, or about 10% of the total volume of the surface coating.

In some instances, the weight of the hydrophilic polymer (e.g. PVA or PEG) per total weight of the surface coating is from about 1% to about 50%. In some instances, the weight of the hydrophilic polymer (e.g. PVA or PEG) per total weight of the surface coating is from about 1% to about 40%. In some instances, the weight of the hydrophilic polymer (e.g. PVA or PEG) per total weight of the surface coating is from about 1% to about 30%. In some instances, the weight of the hydrophilic polymer (e.g. PVA or PEG) per total weight of the surface coating is from about 1% to about 20%. In some instances, the weight of the hydrophilic polymer (e.g. PVA or PEG) per total weight of the surface coating is from about 1% to about 10%.

2) Polyelectrolyte Multilayers

In certain embodiments, the surface coating comprises polyelectrolyte multilayers (PEMs). PEMs described herein comprise a plurality of alternating layers of oppositely charged polymers (i.e., polyelectrolytes). The oppositely charged polymers described herein comprise a combination of a positively charged polyelectrolyte (also referred to herein as a polycation) and a negatively charged polyelectrolyte (also referred to herein as a polyanion).

Exemplary polycations include, but are not limited to, poly(L-lysine) (PLL), poly(L-arginine) (PLA), poly(L-ornithine) (PLO), poly(L-histidine) (PLH), polyethyleneimine (PEI), poly[α-(4-aminobutyl)-L-glycolic acid] (PAGA), 2-(dimethylamino)ethyl methacrylate (DMAEMA), N,N-Diethylaminoethyl methacrylate (DEAEMA), and a combination thereof. In some instances, the polycation is PLL. In some instances, the polycation is PLO. In some instances, the polycation is PLH. In some instances, the polycation is PLA.

Exemplary polyanions include, but are not limited to, poly-L-glutamic acid (PLGA), poly-L-aspartic acid (PLAA), poly(acrylic acid), poly(methacrylic acid) (PMAA), poly(styrenesulfonic acid) (PSS), poly(N-isopropylacrylamide) (NIPAM), poly(2-acrylamido-2-methyl-1-propane sulfonic acid) (PAMPS), and a combination thereof. In some instances, the polyanionis PLGA. In some instances, the polyanion is PLAA.

Polyelectrolyte multilayers may be formed by depositing polycations and polyanions in an alternative fashion via layer-by-layer assembly. Polyelectrolyte multilayers described herein include at least one bilayer including a polycation layer and a polyanion layer.

In some embodiments, the PEMs may include from about 1 bilayers to about 100 bilayers. In some embodiments, the PEMs may include from about 1 bilayers about 50 bilayers. In some embodiments, the PEMs may include from about 1 bilayers to about 30 bilayers. In some embodiments, the PEMs may include from about 1 bilayers to about 20 bilayers. In some embodiments, the number of bilayers is 1, 2, 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16. In some embodiments, the number of bilayers is 3. In some embodiments, the number of bilayers is 4. In some embodiments, the number of bilayers is 5. In some embodiments, the number of bilayers is 6. In some embodiments, the number of bilayers is 7. In some embodiments, the number of bilayers is 8. In some embodiments, the number of bilayers is 9. In some embodiments, the number of bilayers is 10. In some embodiments, the number of bilayers is 11. In some embodiments, the number of bilayers is 12. In some embodiments, the number of bilayers is 13. In some embodiments, the number of bilayers is 14. In some embodiments, the number of bilayers is 15. In some embodiments, the number of bilayers is 16. In some embodiments, the number of bilayers is 17. In some embodiments, the number of bilayers is 18. In some embodiments, the number of bilayers is 19. In some embodiments, the number of bilayers is 20.

In some embodiments, the polyelectrolyte multilayers described herein comprise one or more bilayers of positively charged polyelectrolyte(s) and negatively charged polyelectrolyte(s), in which the polycation is selected from PLL, PLO PLH, and PLA, and the polyanion is selected from PLGA and PLAA. In some embodiments, the number of sets ranges from 1 to 100, 3 to 60, from 3 to 50, or from 3 to 30. In some embodiments, the number of sets is greater than 3, 4, 5, 6, 7, 8, 9, 10, 11, 12, 13, 14, 15, 16, 17, 18, 19 or 20. In some embodiments, the number of sets is 3, 4, 5, 6, 7, 8, 9, 10, 11, 12, 13, 14, 15, 16, 17, 18, 19 or 20. In some embodiments, the number of sets is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLL and PLGA. In some embodiments, the number of bilayers of PLL and PLGA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLO and PLGA. In some embodiments, the number of bilayers of PLO and PLGA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLH and PLGA. In some embodiments, the number of bilayers of PLH and PLGA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLA and PLGA. In some embodiments, the number of bilayers of PLA and PLGA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLL and PLAA. In some embodiments, the number of bilayers of PLL and PLAA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLO and PLAA. In some embodiments, the number of bilayers of PLO and PLAA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLH and PLAA. In some embodiments, the number of bilayers of PLH and PLAA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

In some embodiments, the polyelectrolyte multilayers described herein comprise one or more bilayers of PLA and PLAA. In some embodiments, the number of bilayers of PLA and PLAA ranges from 1 to 100, 3 to 60, from 3 to 50, or from 3 to 30. In some embodiments, the number of bilayers is greater than 3, 4, 5, 6, 7, 8, 9, 10, 11, 12, 13, 14, 15, 16, 17, 18, 19 or 20. In some embodiments, the number of bilayers is 3, 4, 5, 6, 7, 8, 9, 10, 11, 12, 13, 14, 15, 16, 17, 18, 19 or 20. In some embodiments, the number of bilayers is in a range of 1-10, 1-8, 1-5, 3-20, 5-20, 10-20, 11-19, 12-18, 13-17, or 14-16.

The thickness of the PEM as a thin film may be in a broad range, for example, in a range from about 30 nm to about 30 μm, or from about 100 nm to about 20 μm. In some embodiments, the thickness is about 100 nm to about 500 nm, about 500 nm to about 1 μm, or about 1 μm to about 10 μm. In some embodiments, the thickness is about 200, 400, 600, 800 nm, or any number in between. In some embodiments, the thickness is about 1, 5, 10, 15 or 20 μm, or any number in between.

A number of methodologies are available for characterizing PEMs. In some embodiments, the methodologies may comprise ellipsometry (thickness), quartz crystal microbalance with dissipation monitoring (mass adsorbed, viscoelasticity), contact angle analysis (surface energy), Fourier transform infrared spectroscopy (functional groups), X-ray photoelectron spectroscopy (chemical composition), scanning electron microscopy (surface structure), and atomic force microscopy (roughness/surface structure).

In some embodiments, PEMs may be deposited by pipetting polyanion or polycation solutions into/onto the dish, either as a mixture or sequentially.

In some embodiments, a PEM is formed on the surface by dip coating. In dip coating, the substrate is immersed in a polyelectrolyte solution for a set amount of time (usually 10-15 min), followed by multiple rinses and immersion in a second polyelectrolyte solution of opposite charge. This process is repeated until the desired number of layers is achieved.

In some embodiments, the PEM is formed on the surface by spray coating. In some embodiments, a polyelectrolyte may be sprayed onto the surface for 3-10 sec followed by a rest/draining period of 10-30 sec, washing of the surface with a water spray for 3-20 sec, an additional rest period of 10 sec, and repeating the cycle with a polyelectrolyte of opposite charge.

In some embodiments, the PEM is formed on the surface by spin coating. Spin coating is a highly controlled method for solution-based coating of a system. A typical spin coating procedure includes spin coating for 10-15 sec, rinsing at least once by “spin coating” water for 15-30 sec and repeating the procedure with the oppositely charged polyelectrolyte. The wash step may not be necessary in spin coating.

3) Surface Coating Construction

Another aspect of the present disclosure features a method for coating a cell culture article using the composition described herein. The method described herein comprises the steps of: (a) providing a cell culture article having a hydrophobic surface; (b) modifying the hydrophobic surface with a treatment; (c) applying a hydrophilic polymer to the modified surface; and (d) sequentially depositing on the hydrophilic polymer alternating layers of polycations and polyanions.

In some embodiments, the treatment described herein is a plasma treatment, corona discharge or UV ozone treatment. In some embodiments, the hydrophobic surface described herein is irradiated or hydrophilized after the treatment. In some embodiments, the hydrophobic surface is hydrophilized after applying the hydrophilic polymer (e.g., PVA) to the surface. In some embodiments, the hydrophilic polymer (e.g., PVA) is covalently linked (i.e., conjugated) to the surface. A cross-linking agent may be used to facilitate the crosslinking (i.e., conjugation). Exemplary cross-linking agents include, but are not limited to, maleic acid, formaldehyde, glutaraldehyde, butanal (butyraldehyde), sodium borate, or a combination thereof.

As described herein, a surface is hydrophilic if a contact angle for a water droplet on the surface is less than 90 degrees (the contact angle is defined as the angle passing through the drop interior). Embodiments include hydrophilic surfaces with a contact angle from 90 to 0 degrees; Artisans will immediately appreciate that all ranges and values between the explicitly stated bounds are contemplated, with, e.g., any of the following being available as an upper or lower limit: 90, 80, 70, 60, 50, 40, 30, 20, 10, 5, 2, 0 degrees.

In some embodiments, the substrate described herein comprises (polyanion/polycation)ₙ/PVA, wherein the polyanion/polycation is selected from PLGA/PLL, PLAA/PLL, PLGA/PLA, PLAA/PLA, PLGA/PLO, PLAA/PLO, PLGA/PLH and PLAA/PLH, and n is an integer number ranging from 1 to 20, optionally 1, 2, 3, 4, 5, 6, 7, 8, 9, 10, 11, 12, 13, 14, 15, 16, 17, 18, 19, 20. In some embodiments, n is in a range of 1-10, 1-8, 1-5, 3-20, 5-20, 10-20, 11-19, 12-18, 13-17, or 14-16.

In some embodiments, the substrate described herein comprises (polycation/polyanion)ₙ/PEG, wherein the polycation/polyanion is selected from PLL/PLGA, PLL/PLAA, PLA/PLGA, PLA/PLAA, PLO/PLGA, PLO/PLAA, PLH/PLGA and PLH/PLAA, and n is an integer number ranging from 1 to 20, optionally 1, 2, 3, 4, 5, 6, 7, 8, 9, 10, 11, 12, 13, 14, 15, 16, 17, 18, 19, 20. In some embodiments, n is in a range of 1-10, 1-8, 1-5, 3-20, 5-20, 10-20, 11-19, 12-18, 13-17, or 14-16.

In some embodiments, the substrate described herein polycation (polyanion/polycation)ₙ/PEG-acrylate, wherein the polyanion/polycation is selected from PLGA/PLL, PLAA/PLL, PLGA/PLA, PLAA/PLA, PLGA/PLO, PLAA/PLO, PLGA/PLH and PLAA/PLH, and n is an integer number ranging from 1 to 20, optionally 1, 2, 3, 4, 5, 6, 7, 8, 9, 10, 11, 12, 13, 14, 15, 16, 17, 18, 19, 20. In some embodiments, n is in a range of 1-10, 1-8, 1-5, 3-20, 5-20, 10-20, 11-19, 12-18, 13-17, or 14-16.

In some embodiments, the substrate described herein polyanion (polycation/polyanion)ₙ/PVP, wherein the polycation/polyanion is selected from PLL/PLGA, PLL/PLAA, PLA/PLGA, PLA/PLAA, PLO/PLGA, PLO/PLAA, PLH/PLGA and PLH/PLAA, and n is an integer number ranging from 1 to 20, optionally 1, 2, 3, 4, 5, 6, 7, 8, 9, 10, 11, 12, 13, 14, 15, 16, 17, 18, 19, 20. In some embodiments, n is in a range of 1-10, 1-8, 1-5, 3-20, 5-20, 10-20, 11-19, 12-18, 13-17, or 14-16.

The surface coating described herein can be dehydrated or hydrated. In some embodiments, the surface coating is in a dehydrated state. In other embodiments, the surface coating is in a hydrated state. As used herein, a “dehydrated state” and a “hydrated state” each refers to a volume of an aqueous solution (e.g., water) in reference to the total volume of the surface coating. In the dehydrated state, the volume of the aqueous solution (e.g., water) is less than 20%, less than 15%, less than 10%, less than 5%, less than 1%, or less than 0.5% of the total volume of the surface coating. In a hydrated state, the volume of the aqueous solution (e.g., water) is at least 20%, at least 30%, at least 40%, at least 50%, at least 60%, or higher of the total volume of the surface coating.

In some embodiments, the surface coating described herein comprises an aqueous solution (e.g., water). In some cases, the aqueous solution (e.g., water) is from about 1% to about 60% by weight of the total weight of the surface coating. In some cases, the aqueous solution (e.g., water) is from about 1% to about 50% by weight, from about 1% to about 40% by weight, from about 1% to about 30% by weight, from about 1% to about 20% by weight, from about 10% to about 60% by weight, from about 10% to about 50% by weight, from about 10% to about 40% by weight, from about 10% to about 30% by weight, from about 10% to about 20% by weight, from about 20% to about 60% by weight, from about 20% to about 50% by weight, from about 20% to about 40% by weight, or from about 30% to about 60% by weight of the total weight of the surface coating.

In some embodiments, the surface coating further comprises a filler. In some instances, the filler comprises a mineral filler such as but not limited to silica, alumina, calcium carbonate, or silicone resin.

Each of polycations and polyanions, and absorbent polymer may be dissolved in an aqueous solution for use in the present disclosure. The aqueous solution is free, or substantially free, of organic solvents. It will be understood that some minor amounts of organic solvents may be present in the aqueous solution, for example as a result some organic solvent remaining in the polymer after polymerization. As used herein, “substantially free,” as it relates to an organic solvent in an aqueous solution, means that the aqueous solution comprises less than 1% of the organic solvent by weight. In many embodiments, the aqueous solution contains less than 0.8%, less than 0.5%, less than 0.2% or less that 0.1% of an organic solvent.

Each of polycations and polyanions, and absorbent polymer may be dissolved in an aqueous solution at any suitable concentration for the purposes of coating.

### Cell Culture Systems

The cell culture system of the present disclosure comprises a cell culture article having a surface coated with the surface coating described herein.

The cell culture article described herein can be made of any suitable plastic and the like. In certain embodiments, the cell culture article is made of a material comprising at least one of polystyrene, polyethylene terephthalate, polycarbonate, polyvinylpyrrolidone, polybutadiene, polyvinylchloride, polyethylene oxide, polypyrroles, and polypropylene oxide.

In some embodiments, the cell culture system further comprises cells. In some embodiments, the cells are derived from cell lines. In some embodiments, the cells are mammalian cells. In some embodiments, the cells are human cells. In some embodiments, the cells are tissue cells, immune cells, endothelial cells, stem cells, epithelial cells, mesenchymal cells, mesothelial cells, cancer cells or tumor-associated cells. In some embodiments, the cell culture system further comprises a culture media.

The cell culture systems disclosed herein enable not only cell attachment and growth, but also the viable harvest of cultured cells (e.g. 3D cell culture, tissue and organs). According to some embodiments of the present disclosure, the cell culture systems can be used to harvest viable cells, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. In some embodiments, cells can be released from the cell culture systems with or without using a cell dissociation enzyme, for example, trypsin, TrypLE, or Accutase.

### Methods and Uses Thereof

### Methods for Culturing Cells

Without being bound to any particular theory, it is believed that the surface coating disclosed herein enables robust multiplication and/or stable maintenance of cells. The present disclosure thus provides a method for culturing cells. The method comprises the steps of: (a) providing a cell culture article having a surface coated with the surface coating of the present disclosure; (b) seeding cells on the coated surface; and (c) culturing the cells under a suitable medium. In some embodiments, the cells are cultured for a sufficient period of time to form spheroids. In preferred embodiments, the spheroids are 3D spheroids. In some embodiments, the spheroids described herein are generated via single cell proliferation. In some embodiments, the spheroids described herein are generated via single cell proliferation without cell agglomeration. In some embodiments, the spheroids have uniform size.

In some embodiments, the cells described herein may be derived from a cell line, a tissue biopsy or a liquid biopsy. In some embodiments, the cells are mammalian cells. In some embodiments, the cells are human cells. In some embodiments, the cells are tissue cells, immune cells, endothelial cells, stem cells, epithelial cells, mesenchymal cells, mesothelial cells, cancer cells or tumor-associated cells.

In some embodiments, the cells described herein are stem cells such as mesenchymal stem cells (MSCs) or pluripotent stem cells (PSCs) including embryonic stem cells (ESCs) and induced pluripotent stem cells (iPSCs).

In some embodiments, the cells described herein are cancer cells. Exemplary cancer described herein includes, but is not limited to, acute lymphatic cancer, acute myeloid leukemia, alveolar rhabdomycosarcoma, bone cancer, brain cancer, breast cancer, cancer of the anus, anal canal or anorectum cancer, cancer of the eye, cancer of the intrahepatic bile duct cancer, cancer of the joints, cancer of the neck, gallbladder or pleura cancer, cancer of the nose, nasal cavity or middle ear, cancer of the oral cavity, cancer of the vulva, chronic lymphatic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum cancer, omentum and mesentary cancer, pharynx cancer, prostate cancer, rectal cancer, renal cancer, skin cancer, small intestine cancer, soft tissue cancer, stomach cancer, testicular cancer, thyroid cancer, ureter cancer, and urinary bladder cancer.

In some embodiments, the cells described herein are tumor-associated cells. Exemplary tumor-associated cells include, but are not limited to, tumor cell clusters, tumor infiltrating lymphocytes (TILs), cancer associated macrophage-like cells (CAMLs), tumor-associated macrophages (TAMs), tumor-associated monocyte/macrophage lineage cells (MMLCs), cancer stem cells, tumor microemboli, tumor-associated stromal cells (TASC), tumor-associated myeloid cells (TAMCs), tumor-associated regulatory T cells (Treg), cancer-associated fibroblasts (CAFs), tumor-derived endothelial cells (TECs), tumor-associated neutrophils (TAN), tumor-associated platelets (TAP), tumor-associated immune cells (TAI), myeloid-derived suppressor cells (MDSC), and a combination thereof.

Exemplary cells include low-density cells, single cells, rare cells, or a combination thereof. Low-density cells can be cells when seeded, are less than 5000 per cm² on the substrate, e.g., no more than about any of 1, 5, 10, 20, 50, 100, 200, 300, 500, 1000, 2000, 3000, 4000, or 4500 per cm² on the substrate.

In some embodiments, seeding the isolated cells in step (c) comprises plating the cells at a density of between one cell and 10 cells per cm² on the substrate surface (i.e. cell growth surface). In some embodiments, seeding the isolated cells in step (c) comprises plating the cells at a density of between 10 cells and 100 cells per cm² on the substrate surface. In some embodiments, seeding the isolated cells in step (c) comprises plating the cells at a density of between 100 cells and 1000 cells per cm² on the substrate surface.

In some embodiments, the cells are cultured for a period of time ranging from about 2 days to about 5 weeks, such as from about 3 to about 14 days, for example about 7 days. In some embodiments, the cells are cultured for 3 days and the spheroids have an average diameter ranging from about 40 μm to about 200 μm.

Any suitable culture medium can be employed in the methods of exemplary embodiments. Exemplary culture medium includes, but is not limited to, Dulbecco's modified Eagle's medium (DMEM), epidermal growth factor (EGF) and/or basic fibroblast growth factor (bFGF), a mixture of Dulbecco's modified Eagle's medium (DMEM), supplemented with B27 supplement, epidermal growth factor (EGF) and basic fibroblast growth factor (bFGF).

### Method of Preparing Single-Cell-Derived Spheroids

In another aspect, the present disclosure provides a provides a method of preparing a single-cell derived spheroid, the method comprising the steps of: (a) providing a cell culture article having a surface coated with the surface coating of the present disclosure; (b) seeding cells on the coated surface; and (c) culturing the cells under a suitable medium for a sufficient period of time to form spheroids, in which the spheroids are single-cell derived. The spheroids described herein are generated via single cell proliferation. In some embodiments, the spheroids have uniform size. In some embodiments, the single-cell-derived clones are semi-attached or loosely attached on the substrate of the present disclosure.

In some embodiments, the cells are derived from cell lines. In some embodiments, the cells are mammalian cells. In some embodiments, the cells are human cells. In some embodiments, the cells are tissue cells, immune cells, endothelial cells, stem cells, epithelial cells, mesenchymal cells, mesothelial cells, cancer cells or tumor-associated cells.

In some embodiments, the cells are stem cells such as mesenchymal stem cells (MSCs) or pluripotent stem cells (PSCs) including embryonic stem cells (ESCs) and induced pluripotent stem cells (iPSCs).

In some embodiments, the cells are cancer cells. In some embodiments, the cells are cancer cells. In certain embodiments, the cancer cells are isolated from human primary tumor tissue. In certain embodiments, the cancer cells are isolated from a blood sample of a cancer patient. Exemplary cancer described herein includes, but is not limited to, acute lymphatic cancer, acute myeloid leukemia, alveolar rhabdomycosarcoma, bone cancer, brain cancer, breast cancer, cancer of the anus, anal canal or anorectum cancer, cancer of the eye, cancer of the intrahepatic bile duct cancer, cancer of the joints, cancer of the neck, gallbladder or pleura cancer, cancer of the nose, nasal cavity or middle ear, cancer of the oral cavity, cancer of the vulva, chronic lymphatic leukemia, chronic myeloid cancer, colon cancer, esophageal cancer, cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum cancer, omentum and mesentary cancer, pharynx cancer, prostate cancer, rectal cancer, renal cancer, skin cancer, small intestine cancer, soft tissue cancer, stomach cancer, testicular cancer, thyroid cancer, ureter cancer, and urinary bladder cancer.

In some embodiments, the cells are tumor-associated cells. Exemplary tumor-associated cells include, but are not limited to, tumor cell clusters, tumor infiltrating lymphocytes (TILs), cancer associated macrophage-like cells (CAMLs), tumor-associated macrophages (TAMs), tumor-associated monocyte/macrophage lineage cells (MMLCs), cancer stem cells, tumor microemboli, tumor-associated stromal cells (TASC), tumor-associated myeloid cells (TAMCs), tumor-associated regulatory T cells (Treg), cancer-associated fibroblasts (CAFs), tumor-derived endothelial cells (TECs), tumor-associated neutrophils (TAN), tumor-associated platelets (TAP), tumor-associated immune cells (TAI), myeloid-derived suppressor cells (MDSC), and a combination thereof.

Exemplary cells include low-density cells, single cells, rare cells, or a combination thereof. Low-density cells can be cells when seeded, are less than 5000 per cm² on the substrate, e.g., no more than about any of 1, 5, 10, 20, 50, 100, 200, 300, 500, 1000, 2000, 3000, 4000, or 4500 per cm² on the substrate.

In some embodiments, seeding the isolated cells in step (c) comprises plating the cells at a density of between one cell and 10 cells per cm² on the substrate surface (i.e. cell growth surface). In some embodiments, seeding the isolated cells in step (c) comprises plating the cells at a density of between 10 cells and 100 cells per cm² on the substrate surface. In some embodiments, seeding the isolated cells in step (c) comprises plating the cells at a density of between 100 cells and 1000 cells per cm² on the substrate surface.

In some embodiments, the culturing step occurs over a period of 2-8 days (e.g., 2, 3, 4, 5, 6, 7, or 8 days). In other embodiments, the culturing step culturing step occurs over a period of 7-14 days (e.g., 7, 8, 9, 10, 11, 12, 13, or 14 days). In other embodiments, the culturing step culturing step occurs over a period of 1-4 weeks (e.g., 1, 2, 3, or 4 weeks). In some embodiments, the cells are cultured for 3 days and the spheroids have an average diameter ranging from about 40 μm to about 200 μm.

Any suitable culture medium can be employed in the methods of exemplary embodiments. Exemplary culture medium includes, but is not limited to, Dulbecco's modified Eagle's medium (DMEM), epidermal growth factor (EGF) and/or basic fibroblast growth factor (bFGF), a mixture of Dulbecco's modified Eagle's medium (DMEM), supplemented with B27 supplement, epidermal growth factor (EGF) and basic fibroblast growth factor (bFGF).

In some embodiments, the size of a single-cell derived spheroid less than 200 μm in diameter. In some embodiments, the size of a single-cell derived spheroid less than 150 μm in diameter. In some embodiments, the size of a single-cell derived spheroid is about 40, 50, 60, 70, 80, 90, 100, 110, 120, 130 or 140 μm in diameter.

In certain embodiments, the single-cell derived spheroid may be used for screening a therapeutic agent. In certain embodiments, a method of screening a therapeutic agent comprises: (a) applying a test substance to the single-cell derived spheroid generated thereof; and (b) evaluating an effect of the test substance on the single-cell derived spheroid. In some embodiments, the effect of the test substance is analyzed with an imaging system, e.g., to analyze the biochemical activity and/or the expression levels of a gene or a protein.

In some embodiments, the single-cell derived spheroid generated thereof is a tumor spheroid. In some embodiments, the test substance described herein is a chemotherapeutic drug, such as a cytotoxic or cytostatic chemotherapeutic drug. In some embodiments, the therapeutic agent is an immune checkpoint inhibitor, such as an immune checkpoint inhibitor. In some embodiments, the therapeutic agent is a nucleic acid drug. In some embodiments, the therapeutic agent is a therapeutic cell composition, including, but not limited to, T cells, natural killer (NK) cells, and dendritic cells.

In some embodiments, the cells are cultured for a period of time ranging from about 2 days to about 5 weeks, such as from about 3 to about 14 days, for example about 7 days. In some embodiments, the cells are cultured for 3 days and the at least one 3D spheroid has an average diameter ranging from about 40 μm to about 200 μm.

In some aspects, provided herein is a single-cell-derived spheroid (e.g., tumor spheroid) generated according to any one of the culture methods employing the cell culture systems described herein. In some aspects, there is provided a library of single-cell-derived spheroids (e.g., tumor spheroids) derived according to any one of the culture methods employing the cell culture systems described herein.

### Method of Isolating Single-Cell-Derived Clones

Single-cell-derived clone has gained increasing importance as genome editing techniques have entered routine laboratory practice. Limiting dilution, the traditional method for isolating single cells, relies on statistical probabilities for monoclonality that can vary significantly with slight changes to protocols. The technique, while highly inefficient at isolating single cells, preserves cell viability. Conversely, flow cytometry can provide single cell clones with high efficiency but negatively affects cell viability. A common trait of these platforms is that they generally start with a suspension containing a large number of cells that are ‘individualized’ by random confinement in microstructures. Both of these methods are impractical when the cell population is small as they generate considerable cell loss during mixing and/or transfer. The method of the invention provides an efficient alternative for isolating viable single cell clones. In some embodiments, the method does not require individual confinement of cells in microstructures.

In some embodiments, there is provided herein a method of isolating a single-cell-derived clone. The method described herein comprises: 1) culturing a heterogeneous population of cells using a cell culture article having a surface coated with the composition of the present disclosure to obtain a plurality of cell clones comprising a single-cell-derived clone; and 2) isolating the single-cell-derived clone from the cell culture article.

In some embodiments, the heterogeneous population of cells comprises adherent cells. In some embodiments, the heterogeneous population of cells comprises non-adherent cells. In some embodiments, the heterogeneous population of cells comprises cells isolated from a cell line. In some embodiments, the heterogeneous population of cells comprises cells isolated from a liquid biopsy of a subject. In some embodiments, the heterogeneous population of cells comprises cells isolated from a tissue biopsy of a subject. In some embodiments, the heterogeneous population of cells comprises cells that have been genetically engineered. In some embodiments, the heterogeneous population of cells comprises cells that have been engineered to comprise a genetic mutation. In some embodiments, the heterogeneous population of cells comprises cells that have been engineered to comprise a heterologous nucleotide sequence.

In some embodiments, the single-cell-derived clones are semi-attached or loosely attached on the coated surface disclosed herein.

In some embodiments, no cell debris is observed in the cell culture system after 7 or more days of cultivation.

In some embodiments, the culturing step occurs over a period of 2-8 days (e.g., 2, 3, 4, 5, 6, 7, or 8 days). In other embodiments, the culturing step culturing step occurs over a period of 7-14 days (e.g., 7, 8, 9, 10, 11, 12, 13, or 14 days). In other embodiments, the culturing step culturing step occurs over a period of 1-4 weeks (e.g., 1, 2, 3, or 4 weeks).

In some embodiments, the single-cell-derived clone forms a single-cell-derived spheroid. In some embodiments, the single-cell-derived clone has a diameter of from about 40 μm to about 200 μm. In some embodiments, the single-cell-derived clone has a diameter of from about 50 μm to about 150 μm. In some cases, the single-cell-derived clone has a diameter of from about 50 μm to about 120 μm, from about 50 μm to about 100 μm, from about 50 μm to about 80 μm, from about 50 μm to about 60 μm, from about 80 μm to about 150 μm, from about 80 μm to about 120 μm, from about 80 μm to about 100 μm, from about 100 μm to about 200 μm, from about 100 μm to about 150 μm, or from about 100 μm to about 120 μm.

In some instances, the single-cell-derived clones form a single-cell-derived spheroid. In some cases, the spheroid comprises from about 8 to about 1000 cells. In some cases, the spheroid comprises from about 8 to about 800 cells, from about 8 to about 500 cells, from about 8 to about 400 cells, from about 8 to about 300 cells, from about 8 to about 200 cells, from about 8 to about 100 cells, from about 10 to about 1000 cells, from about 10 to about 800 cells, from about 10 to about 500 cells, from about 10 to about 400 cells, from about 10 to about 300 cells, from about 10 to about 200 cells, from about 10 to about 100 cells, from about 50 to about 1000 cells, from about 50 to about 800 cells, from about 50 to about 500 cells, from about 50 to about 400 cells, from about 50 to about 300 cells, from about 50 to about 200 cells, from about 100 to about 1000 cells, from about 100 to about 800 cells, from about 100 to about 500 cells, from about 100 to about 400 cells, from about 100 to about 300 cells, from about 300 to about 1000 cells, from about 300 to about 800 cells, from about 300 to about 500 cells, from about 500 to about 1000 cells, or from about 500 to about 800 cells.

In some embodiments, at least 10% of the cells disposed on the coated surface forms single-cell-derived spheroids. In some embodiments, at least 20% of the cells disposed on the coated surface forms single-cell-derived spheroids. In some embodiments, at least 30% of the cells disposed on the coated surface forms single-cell-derived spheroids. In some embodiments, at least 40% of the cells disposed on the coated surface forms single-cell-derived spheroids. In some embodiments, at least 50% of the cells disposed on the coated surface forms single-cell-derived spheroids. In some embodiments, at least 60% of the cells disposed on the coated surface forms single-cell-derived spheroids. In some embodiments, at least 70% of the cells disposed on the coated surface forms single-cell-derived spheroids.

In some embodiments, the method described herein further comprises analyzing the single-cell-derived clone, thereby obtaining a characteristic of the single cell. In some instances, the step of analyzing the single-cell-derived clone comprises subjecting the single-cell-derived clone to sequencing analysis. In some embodiments, the analyzing step comprises performing a genotyping analysis. In some embodiments, the genotyping analysis is a PCR-based analysis. In some embodiments, the genotyping analysis is an array hybridization-based analysis. In some embodiments, the analyzing step comprises analyzing a copy number variation. In some embodiments, the analyzing step comprises analyzing a genetic mutation. In some embodiments, the analyzing step comprises analyzing a single nucleotide polymorphism.

In some embodiments, the step of analyzing the single-cell-derived clone comprises subjecting the single-cell-derived clone to proteomic analysis. Exemplary proteomic analysis include gel electrophoresis such as polyacrylamide gel electrophoresis (PAGE), sodium dodecyl sulfate polyacrylamide gel electrophoresis (SDS-PAGE), two-dimensional gel electrophoresis, or capillary electrophoresis; high-performance liquid chromatography (HPLC); and affinity chromatography.

In some embodiments, the characteristic of the single cell comprises one or more of: a genotype, an epigenetic profile, an expression level of a gene or a protein, a response to a drug, a drug resistance profile, or a metastatic potential.

In some aspects, provided herein is a single-cell-derived clone generated according to any one of the culture methods employing the cell culture systems described herein. In some aspects, there is provided a library of single-cell-derived clones derived according to any one of the culture methods employing the cell culture systems described herein.

Generation of Patient-Derived Tumoroid Xenografts

In another aspect, the present disclosure provides a method for generating patient-derived tumoroid-based xenograft. Patient-derived tumoroids can be generated via in vitro growth of tumor cells derived from patient blood, tissue (e.g. bladder, stomach, breast, pancreas, colon, or lung) or cell lines. Patient-derived tumoroid-based xenografts can be established by the direct transfer of tumoroids into highly immunodeficient mice and then maintained by passaging from mouse to mouse. The xenograft model is useful for biomedical translational research, and once validated, it can be used as a translational preclinical model for efficacy screening in cancer drug development.

In some embodiments, provided herein is a method of generating a patient-derived tumor xenograft animal model, comprising: a) culturing a plurality of cells comprising tumor cells derived from a patient using any of the cell culture systems provided herein to obtain a plurality of tumoroids; b) isolating a tumoroid from the 3D cell culture system; and 3) inoculating the isolated tumoroid into a non-human animal, thereby generating the patient-derived tumor xenograft animal model.

In some embodiments, the plurality of tumor cells are obtained from a primary tissue of the patient. In some embodiments, the plurality of tumor cells are obtained from the blood of the patient. In some embodiments, the tumor cells derived from a patient are grown as animal model primary xenografts prior to culturing on the 3D cell culture system.

In some embodiments, the non-human animal is immunodeficient. In some embodiments, the non-human animal is a mouse.

In some embodiments, the tumor cells are circulating tumor cells (CTCs) derived from a solid tumor. In other instances, the tumor cells are CTCs derived from a hematologic malignancy. In some embodiments, the patient has a metastatic cancer.

In some embodiments, the culturing step comprises expansion of the tumor cells by 10 to 100-fold (e.g., 10-, 20-, 30-, 40-, 50-, 60-, 70-, 80-, 90-, or 100-fold) within one week.

Methods that employ non-adherent conditions for 3D cell culture include the hanging drop method (Kelm et al. Biotechnol. Bioeng. 83, 173-180 (2003)), rotating bioreactor (Zhau, et al. In Vitro Cell. Dev. Biol. Anim. 33, 375-380 (1997)), magnetic levitation (Souza et al. Nat. Nanotechnol. 5, 291-296 (2010)). However, some of the most widely used non-adherent techniques do not represent a true 3D cell culture that mimics tumor formation in vivo. When tens-of-thousands cells are aggregated into a spheroid (i.e., a mass with spherical shape) such as in a hanging drop, reactor or U-bottom plates, an extensive central necrotic core forms over a few hours due to the lack of nutrient and oxygen penetration beyond a 200 μm depth. Extended central necrosis is a rare phenomenon in real cancers. This nonphysiologically-relevant cancer representation is exacerbated by the lack of progressive tumor development via cell division and the lack of interaction with an appropriate extracellular matrix (ECM). In some embodiments, the 3D cell culture systems provided herein are able to maintain the size of a cell spheroid/tumoroid around 100 μm and induce its division to a smaller spheroid as cells continue to proliferate over time.

In some embodiments, the tumoroid has a diameter from about 50 μM to about 150 μM. In some embodiments, the tumoroid has a diameter from about 100 μM to about 150 μM. In some cases, the tumoroid has a diameter of about 50 μM, about 60 μM, about 70 μM, about 80 μM, about 90 μM, about 100 μM, about 110 μM, about 120 μM, about 130 μM, about 140 μM, or about 150 μM. In some embodiments, the average size of the tumoroids is about 150 μM after 8 days of culturing.

In some embodiments, the culturing step occurs over a period of 7-14 days (e.g., 7, 8, 9, 10, 11, 12, 13, or 14 days). In some embodiments, the size of the tumoroid cultured for more than 7 days is maintained within the range of from about 50 μM to about 150 μM in diameter. In some cases, the tumoroid has a diameter of about 50 μM, about 60 μM, about 70 μM, about 80 μM, about 90 μM, about 100 μM, about 110 μM, about 120 μM, about 130 μM, about 140 μM, or about 150 μM.

In some embodiments, the plurality of cells further comprises tumor-associated cells derived from the patient. In some embodiments, the tumor-associated cells comprise tumor-associated stromal cells. In some embodiments, the plurality of tumoroids comprise tumor cells and tumor-associated cells. In some embodiments, the tumoroid is derived from a single cell.

In some embodiments, the cancer is a solid tumor or a hematologic malignancy. In some cases, the cancer is colorectal, breast, pancreatic, head and neck, bladder, ovarian, stomach, or prostate cancer.

In some embodiments, the method of making a patient-derived tumoroid provides tumoroids at a yield ratio (e.g., yield from CTCs) of between 0.5 and 1. In some embodiments, the method of making a patient-derived tumoroid provides tumoroids at a yield ratio (e.g., yield from CTCs) of between 0.6 and 1. In some embodiments, the method of making a patient-derived tumoroid provides tumoroids at a yield ratio (e.g., yield from CTCs) of between 0.7 and 1. In some embodiments, the method of making a patient-derived tumoroid provides tumoroids at a yield ratio (e.g., yield from CTCs) of between 0.8 and 1.

In some aspects, there is provided herein a patient-derived tumor xenograft model prepared according any one of the methods employing the cell culture systems described herein. In some embodiments, the patient-derived tumor xenograft model is capable of spontaneous metastasis. In some embodiments, the xenograft model can be propagated in vivo by injecting patient-derived tumor cells isolated from one xenograft model into another non-human animal.

In some aspects, there is provided herein a method of analyzing the in vivo activity of a therapeutic agent (e.g., an anti-cancer drug), comprising administering the therapeutic agent (e.g., the anti-cancer drug) to the patient-derived tumor xenograft animal model prepared according to any one of the methods described herein, and analyzing the effect of the therapeutic agent on the patient-derived tumor xenograft animal model.

In some aspects, there is provided herein a biobank comprising a plurality of different tumoroids prepared according to any one of the methods employing the cell culture systems described herein.

Generation of 3D Co-Cultures In Vitro

In another aspect, the present disclosure provides a method for generating 3D co-culture tumor model derived from tumor cells and tumor-associated stromal or immune cells (e.g. fibroblasts, endothelial cells and immune cells), in particular, patient-derived 3D co-culture tumor model derived from patient-derived tumor cells and tumor-associated stromal cells (e.g. fibroblasts, endothelial cells and/or immune cells). 3D co-culture tumor model provides an improved mimicry of in vivo tumor microenvironments that is useful for basic research on tumor microenvironment and establishment of in vitro drug screening models for cancer therapy and cancer immunotherapy.

In some embodiments, provided herein is a method for generating a 3D co-culture tumor model derived from tumor cells and tumor-associated stromal or immune cells (e.g. fibroblasts, endothelial cells and/or immune cells), wherein the method comprises a) culturing a plurality of cells comprising tumor cells and tumor-associated stromal cells on any of the 3D culture systems provided herein to obtain a plurality tumoroids comprising both tumor cells and tumor-associated stromal cells. In some embodiments, the tumor cells and tumor-associated stromal cells form direct cell-cell contacts within the tumoroids. In some embodiments, the method comprises aggregating tumor cells and tumor-associated stromal cells (e.g., by culturing the mixed cell population.

In some embodiments, the stromal cells comprise fibroblasts, endothelial cells, or mesenchymal stem cells. In some embodiments, the immune cells comprise myeloid-derived suppressor cells (MDSCs), tumor associated macrophages, neutrophils, tumor-infiltrating lymphocytes, T cells, B cells, dendritic cells, or any other tumor-associated immune cells.

In some embodiments, the 3D co-culture tumor models provided herein provide a valuable tool to study the cytotoxic effect of anticancer drugs on normal cells. In some embodiments, a method provided herein comprises evaluating the effect of an anticancer drug on tumor cells (e.g., highly proliferative cells) vs. on normal, non-tumor cells within the co-culture.

Generation of 3D Cell Culture for Hepatocytes

In another aspect, the present disclosure provides a method for generating 3D cell culture model for hepatocytes. 3D cell culture exhibits superior liver-specific functions over the conventional 2D cell culture in evaluating hepatobiliary drug disposition and drug-induced hepatotoxicity due to the in vivo-like physiological condition recapitulated by 3D model. 3D liver cell culture is useful for tissue engineering and drug development.

Once hepatocytes are isolated from the liver and are grown in conventional primary cultures, the activity of these important enzymes is rapidly lost. This loss is particularly prominent for rat hepatocytes which lose 80% of their CYP activity in the first 24 hours of culture (Paine, A J, In: Berry, M N et al. (eds.), The Hepatocyte Review, Kluwer Academic Publishers, Netherlands, pp. 411-420, 2000).

In some embodiments, provided herein is a method for culturing hepatocytes on any one of the 3D culture systems described herein, wherein the hepatocytes maintain one or more liver-specific gene expressions and functions of primary hepatocytes, such as albumin secretion, viral infectivity, and/or cytochrome 3 P450 (CYP) enzyme activity. CYPs are a family of enzymes, localized to the cytoplasmic side of the endoplasmic reticulum of the liver cell, that catalyze the oxidation of organic compounds, resulting in increased water solubility which promotes excretion from the cell. In some embodiments, the hepatocytes cultured on any of the 3D culture systems described herein maintain gene expression and function of one or more genes involved in normal drug metabolism for a culturing period of at least 5 days, at least 7 days, at least 10 days, at least 14 days, or at least 21 days.

In some embodiments, the 3D cell culture platform is used for culture of primary hepatocytes. In some embodiments, the primary hepatocytes are isolated from a liver biopsy of a human or other mammal. In some embodiments, the 3D cell culture platform is used for culture of fetal liver cells.

In some embodiments, the 3D cell culture platform is used for culturing an immortalized cell line. In some embodiments, the cell line expresses one or more Phase 1/II xenobiotic drug metabolism genes and/or hepatocyte-specific transcripts. In some embodiments, the cells cultured on any of the 3D culture systems described herein maintain gene expression and function of one or more genes involved in normal drug metabolism for a culturing period of at least 5 days, at least 7 days, at least 10 days, at least 14 days, or at least 21 days. In some embodiments, the cells are HepG2, Huh7, or HepaRG.

In some embodiments, provided herein is a method of culturing hepatocytes using any one of the 3D cell culture systems described herein, wherein the hepatocytes maintain hepatic functions in vitro, such as the expression of critical cytochrome P450 (CYP) drug metabolizing enzymes. In some embodiments, the hepatocytes maintain hepatic function over an extended period of time, such as at least 5 days, at least 7 days, at least 10 days, at least 15 days, or at least 20 days.

Culturing hepatocytes using conventional 3D culture systems results in formation of a necrotic core in the cell spheroids due to hypoxia in the center of spheroids greater than 200 μM (Hussein et al. “Three dimensional culture of HepG2 liver cells on a rat decellularized liver matrix for pharmacological studies.” J Biomed Mater Res B Appl Biomater, 2016. 104(2): p. 263-73). In some embodiments, the 3D cell culture systems provided herein are able to maintain the size of a cell spheroid (e.g., a liver cell spheroid) around 100 μm and induce its division to a smaller spheroid as cells continue to proliferate over time.

In some embodiments, the cell spheroid has a diameter of from about 50 μM to about 150 μM. In some embodiments, the cell spheroid has a diameter of from about 100 μM to about 150 μM. In some cases, the cell spheroid has a diameter of about 50 μM, about 60 μM, about 70 μM, about 80 μM, about 90 μM, about 100 μM, about 110 μM, about 120 μM, about 130 μM, about 140 μM, or about 150 μM. In some embodiments, the average size of the cell spheroids is 150 μM after 8 days of culturing. In some embodiments, the size of the cell spheroid cultured for more than 7 days is maintained within the range of from about 50 μM to about 150 μM.

In some embodiments, the method comprises culturing liver cells and nonparenchymal cells (NPCs) on any of the 3D culture systems provided herein. In some embodiments, the nonparenchymal cells include bile duct epithelial cells, liver sinusoidal endothelial cells (LSEC), hepatic stellate cells (HSC) and/or Kupffer 8 cells (KC).

In another embodiment, this invention provides a method for evaluating the metabolism of an agent that is metabolized by mammalian liver cells in vivo, comprising (a) culturing mammalian hepatocytes using any of the 3D culture systems described herein; (b) adding the agent being evaluated to the hepatocyte culture in the culture vessels for a period of time sufficient for enzymes of the hepatocytes to metabolize the agent and converting it to one of more metabolites thereof; (c) identifying the presence of, or measuring the concentration of, the one or more metabolites in the medium or cells of the culture, thereby evaluating the metabolism of the agent.

Generation of Stem Cell Spheroids In Vitro

In another aspect, the present disclosure provides a method for generating stem cell spheroids in vitro. Stem cells described herein comprise mesenchymal stem cells (MSCs) and pluripotent stem cells (PSCs) including embryonic stem cells (ESCs) and induced pluripotent stem cells (iPSCs). These stem cell spheroids are promising candidates for cell therapies, tissue engineering, high throughput pharmacology screens, and toxicity testing. These applications require large numbers of high quality cells; however, scalable production of MSCs and PSCs has been a challenge. 3D culture system provided herein can allow efficient stem cell proliferation and differentiation of MSCs and PSCs.

In some embodiments, provided herein is a method of 3D cell culture using any one of the 3D culture systems provided herein, wherein the method increases stemness properties and/or proliferation rate of a cell population. In some embodiments, the cells are mesenchymal stem cells or pluripotent stem cells. In some embodiments, the cells are embryonic stem cells or induced pluripotent stem cells.

In some embodiments, the method increases expression of one or more stemness-associated genes, For example, cell-surface CD133 represents one of the biomarkers for stem cell characterization and is associated with multiple cellular characters such as stemness, regeneration, differentiation, and metabolism of diverse cell lineages. In some embodiments, the method increases expression of CD133 in the cells. In some embodiments, the method induces CD133 expression in a population of CD133⁻ cells.

In some embodiments, the method maintains expression of one or more stemness-associated genes for the duration of a culturing period. In some embodiments, the culturing period is at least 7 days, at least 10 days, at least 14 days, or at least 21 days.

Kits

In certain embodiments, disclosed herein is a kit or article of manufacture that comprises a 3D cell culture system described herein. In some instances, the kit is for use in isolating a single cell-derived clone. In some instances, the kit further comprises a package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some cases, the kit further comprises labels listing contents and/or instructions for use, and package inserts with instructions for use, e.g., instructions for culturing a heterogeneous population of cells using a 3D culture system described herein to obtain a plurality of cell clones comprising a single-cell-derived clone. A set of instructions will also typically be included.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the term “comprising” is intended to mean that the methods include the recited steps or elements, but do not exclude others. “Consisting essentially of” shall mean rendering the claims open only for the inclusion of steps or elements, which do not materially affect the basic and novel characteristics of the claimed methods. “Consisting of” shall mean excluding any element or step not specified in the claim. Embodiments defined by each of these transition terms are within the scope of this disclosure.

As used herein, the term “positively charged polyelectrolyte” encompasses a plurality of monomer units or a non-polymeric molecule that comprises two or more positive charges. In some instances, the positively charged polyelectrolyte also encompasses a plurality of monomer units or a non-polymeric molecule that comprise charge positive groups, charge neutral groups, or charge negative groups, with a net charge of being positive.

As used herein, the term “cationic polymer” encompasses a plurality of monomer units or a non-polymeric molecule. In some instances, the cationic polymer is a synthetic polymer. In other instances, the cationic polymer is a natural polymer.

As used herein, the term “cationic polypeptide” refers to a polypeptide comprising two or more positive charges. In some instances, the cationic polypeptide comprises positively charged amino acid residues, negatively charged residues, and polar residues but the net charge of the polypeptide is positive. In some cases, the cationic polypeptide is from 8 to 100 amino acids in length. In some cases, the cationic polypeptide is from 8 to 80, 8 to 50, 8 to 40, 8 to 30, 8 to 25, 8 to 20, 8 to 15, 10 to 100, 10 to 80, 10 to 50, 10 to 40, 10 to 30, 10 to 20, 20 to 100, 20 to 80, 20 to 50, 20 to 40, 20 to 30, 30 to 100, 30 to 80, 30 to 50, 40 to 100, 40 to 80, or 50 to 100 amino acids in length.

As used herein, the term “negatively charged polyelectrolyte” encompasses a plurality of monomer units or a non-polymeric molecule that comprises two or more negative charges. In some instances, the negatively charged polyelectrolyte also encompasses a plurality of monomer units or a non-polymeric molecule that comprise charge positive groups, charge neutral groups, or charge negative groups, with a net charge of being negative.

As used herein, the term “anionic polymer” encompasses a plurality of monomer units or a non-polymeric molecule. In some instances, the anionic polymer is a synthetic polymer. In other instances, the anionic polymer is a natural polymer.

As used herein, the term “anionic polypeptide” refers to a polypeptide comprising two or more negative charges. In some instances, the anionic polypeptide comprises positively charged amino acid residues, negatively charged residues, and polar residues but the net charge of the polypeptide is negative. In some cases, the anionic polypeptide is from 8 to 100 amino acids in length. In some cases, the anionic polypeptide is from 8 to 80, 8 to 50, 8 to 40, 8 to 30, 8 to 25, 8 to 20, 8 to 15, 10 to 100, 10 to 80, 10 to 50, 10 to 40, 10 to 30, 10 to 20, 20 to 100, 20 to 80, 20 to 50, 20 to 40, 20 to 30, 30 to 100, 30 to 80, 30 to 50, 40 to 100, 40 to 80, or 50 to 100 amino acids in length.

As used herein, the term “hydrophilic polymer” encompasses a plurality of monomer units or a non-polymeric molecule that comprise one or more hydrophilic groups. In some instances, the hydrophilic polymer is permeable to an aqueous solution. In other instances, the hydrophilic polymer is impermeable or does not absorb the aqueous solution. In some cases, the hydrophilic polymer encompasses a non-reactive polymer, or a polymer that does not contain a reactive group, e.g., a group that forms covalent bonds with another compound.

As used herein, the term “polymer” includes both homo- and copolymers, branched and unbranched, and natural or synthetic polymers.

As used herein, the term “article” refers to a cell culture article, such as a sheet, film, tube, plate, dish, or a biomedical device. In some instances, a biomedical device is any article that is designed to be used while either in or on tissue (e.g., mammalian tissue) or fluid, preferably in or on human tissue or fluids. Exemplary devices include, but are not limited to, cell culturing dishes, cell culture plates, bioreactors, and the like.

As used herein, immune cells encompass neutrophils, eosinophils, basophils, mast cells, monocytes, macrophages, dendritic cells, natural killer cells, and lymphocytes (B cells and T cells).

Endothelial cells are cells that line the interior surface of blood vessels and lymphatic vessels. Exemplary endothelial cells include high endothelial venules (HEV), endothelium of the bone marrow, and endothelium of the brain.

Epithelial cells are cells that line the outer surfaces of organs and blood vessels, and the inner surfaces of cavities within internal organs. Exemplary epithelial cells include squamous epithelium, cuboidal epithelium, and columnar epithelium.

As used herein, the term “stem cell” encompasses an adult stem cell and an embryonic stem cell. Exemplary stem cells include hematopoietic stem cells, mesenchymal stem cells (MSCs), neural stem cells, epithelial stem cells, skin stem cells, embryonic stem cells (ESCs), and induced pluripotent stem cells (iPSCs).

As used herein, the term “chemically defined medium” refers to an in vitro culture medium in which all of the chemical components are known. A chemically defined medium can include a basal media (such as DMEM, F12, or RPMI 1640, containing amino acids, vitamins, inorganic salts, buffers, antioxidants and energy sources), which is supplemented with recombinant albumin, chemically defined lipids, recombinant insulin and/or zinc, recombinant transferrin or iron, selenium and an antioxidant thiol such as 2-mercaptoethanol or 1-thioglycerol.

As used herein, the term “enriched medium” refers to an in vitro culture medium in which a basal media is further supplemented with growth factors, vitamins, and essential nutrients.

As used herein, the term “semi-attached” and “loosely attached” are used interchangeably and in reference to cultured cells refer to cells that can be detached from the surface of a substrate with gentle agitation, or gentle mechanical force. In some instances, the cells can be detached without the need for a cell dissociation enzyme.

As used herein, the terms “single-cell-derived spheroid” refers to a cluster of cells grown ex vivo and formed in 3D format, which cluster is grown from a single cell disposed on the surface coating.

In some embodiments, therapeutic agents include, but are not limited to, a chemotherapeutic drug, an immune checkpoint inhibitor, a nucleic acid drug, a therapeutic cell composition, or a combination thereof.

In some embodiments, the therapeutic agent is a cytotoxic or cytostatic chemotherapeutic drug. The chemotherapeutic drug can be alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as an actinomycin such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; anti-PD-1 (anti-programmed death-1) therapeutics such as antibodies or compounds (e.g., Nivolumab); thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (Nexavar®), erlotinib (Tarceva®), pazopanib, axitinib, and lapatinib; transforming growth factor-α or transforming growth factor-β inhibitors, and antibodies to the epidermal growth factor receptor such as panitumumab (VECTIBIX®) and cetuximab (ERBITUX®).

In some embodiments, the therapeutic agent is an immune checkpoint inhibitor. The immune checkpoint inhibitor can be CD137, CD134, PD-1, KIR, LAG-3, PD-L1, PDL2, CTLA-4, B7.1, B7.2, B7-DC, B7-H1, B7-H2, B7-H3, B7-H4, B7-H5, B7-H6, B7-H7, BTLA, LIGHT, HVEM, GAL9, TIM-3, TIGHT, VISTA, 2B4, CGEN-15049, CHK 1, CHK2, A2aR, TGF-β, PI3Kγ, GITR, ICOS, IDO, TLR, IL-2R, IL-10, PVRIG, CCRY, OX-40, CD160, CD20, CD52, CD47, CD73, CD27-CD70, CD40, and a combination thereof.

In some embodiments, the therapeutic agent is a nucleic acid drug. The nucleic acid drug can be DNA, DNA plasmid, nDNA, mtDNA, gDNA, RNA, siRNA, miRNA, mRNA, piRNA, antisense RNA, snRNA, snoRNA, vRNA, and a combination thereof. In some embodiments, the therapeutic nucleic acid is a DNA plasmid comprising a nucleotide sequence encoding a gene selected from the group consisting of GM-CSF, IL-12, IL-6, IL-4, IL-12, TNF, IFNy, IFNa, and a combination thereof.

In some embodiments, the therapeutic agent is a therapeutic cell composition. Exemplary therapeutic cell compositions include, but are not limited to T cells, natural killer (NK) cells and dendritic cells.

In some embodiments, the therapeutic agent is a therapeutic antigen-binding molecule composition. Exemplary therapeutic antigen-binding molecule compositions include, but are not limited to monoclonal antibody, bispecific antibody, multispecific antibody, scFv, Fab, VHH/VH, etc.

In some cases, the therapeutic agent comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers.

In some cases, the therapeutic agent comprises a second-line therapy, a third-line therapy, or a fourth-line therapy.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### EXAMPLES

These examples are provided for illustrative purposes only and not to limit the scope of the claims provided herein.

### Example 1: Construction of the Surface Coating of the Invention

(i) PVA or PEG Coated Polystyrene Plate

A tissue culture plate made of polystyrene plastic is first treated by exposing the polystyrene plate to a plasma gas to modify the hydrophobic plastic surface to make it more hydrophilic, followed by depositing a hydrophilic polymer (e.g. PVA or PEG) onto the modified surface of the polystyrene plate to form a PVA or PEG-coated polystyrene plate.

(ii) PVA-Crosslinked Polystyrene Plate

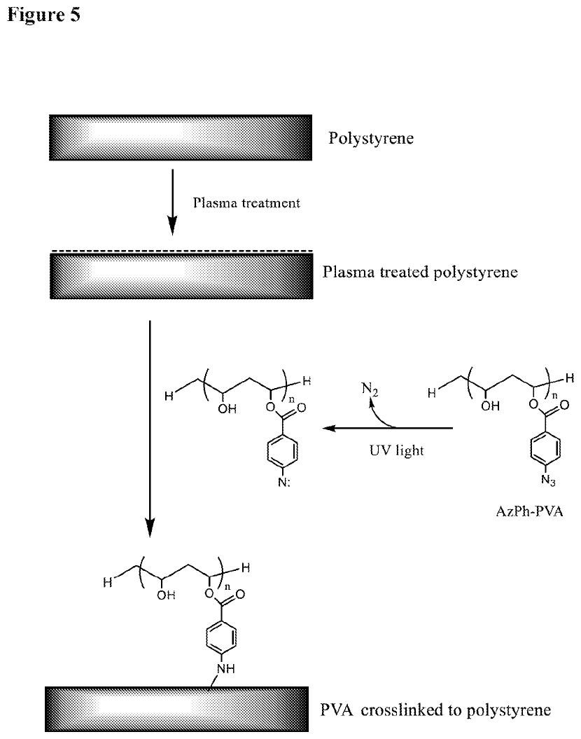

A tissue culture plate made of polystyrene plastic is first treated by exposing the polystyrene plate to an ozone plasma to modify the hydrophobic plastic surface to make it more hydrophilic, followed by addition of a photo-activated azidophenyl-PVA to the plasma treated surface of the polystyrene plate to form a PVA-crosslinked polystyrene plate (shown in FIG. 5). Azidophenyl-derivatized poly(vinyl alcohol) (AzPh-PVA) can be synthesized by coupling —OH groups of PVA to 4-azidobenzoic acid, as reported (J. Nanosci. Nanotechnol., 2009, 9, 230-239).

(iii) PVA-Crosslinked PTFE Plate

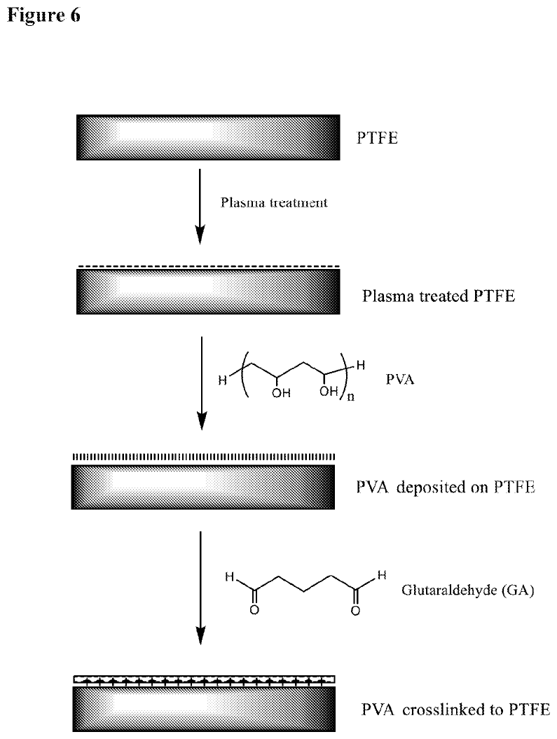

A tissue culture plate made of polytetrafluoroethylene (PTFE) is first treated by exposing the PTFE plate to a plasma gas to modify the hydrophobic plastic surface to make it more hydrophilic, followed by depositing a hydrophilic polymer (e.g. PVA or PEG) onto the modified surface of the PTFE plate. A cross-linking agent, glutaraldehyde (GA), is applied to crosslink PVA to PTFE to form a PVA-crosslinked PTFE plate (shown in FIG. 6)

Buildup of Polyelectrolyte Multilayers

PLL (MW 150K-300K), PLGA (MW 50K-100K), PLO (0.01%) solution, PLH (MW 5K-25K), PLA (MW 15K-70K) are commercially available from Sigma-Aldrich (St. Louis, MO, USA). Both polycation and polyanion are dissolved in Tris-HCl buffer (pH 7.4) and deposited onto the PVA or PEG coated surface after rinsing with Tris-HCl buffer. Each layer of polycation or polyanion is deposited and incubated for 10 min, followed by washing with Tris-HCl buffer 3 times for 2, 1, and 1 min. The PLL/PLGA, PLO/PLGA, PLH/PLGA and PLA/PLGA multilayer films can be fabricated by layer-by-layer self-assembly onto the PVA or PEG coated surface as follows.

PLL/PLGA Multilayers

In some embodiments, the polyelectrolyte multilayers are PLL/PLGA multilayers that can be constructed by sequentially depositing PLL and PLGA on a surface of (i) PVA or PEG-coated polystyrene plate, (ii) PVA-crosslinked polystyrene plate, or (iii) PVA-crosslinked PTFE plate. Each depositing step comprises adding the PLL or PLGA solution to the plate surface, incubated for 10 min and washed 3 times for 2, 1, and 1 min.

In one embodiment, the surface coating composed of (PLGA/PLL)₃/PVA is constructed. In one embodiment, the surface coating composed of (PLGA/PLL)₅/PVA is constructed. In one embodiment, the surface coating composed of (PLGA/PLL)₁₀/PVA is constructed. In one embodiment, the surface coating composed of (PLGA/PLL)₁₅/PVA is constructed. In one embodiment, the surface coating composed of PLL(PLGA/PLL)₃/PVA is constructed. In one embodiment, the surface coating composed of PLL (PLGA/PLL)₅/PVA is constructed. In one embodiment, the surface coating composed of PLL (PLGA/PLL)₁₀/PVA is constructed. In one embodiment, the surface coating composed of PLL (PLGA/PLL)₁₅/PVA is constructed.

PLO/PLGA Multilayers

In some embodiments, the polyelectrolyte multilayers are PLO/PLGA multilayers that can be constructed by sequentially depositing PLO and PLGA on a surface of (i) PVA or PEG-coated polystyrene plate, (ii) PVA-crosslinked polystyrene plate, or (iii) PVA-crosslinked PTFE plate. Each depositing step comprises adding the PLO or PLGA solution to the plate surface, incubated for 10 min and washed 3 times for 2, 1, and 1 min.

In one embodiment, the surface coating composed of (PLGA/PLO)₃/PVA is constructed. In one embodiment, the surface coating composed of (PLGA/PLO)₅/PVA is constructed. In one embodiment, the surface coating composed of (PLGA/PLO)₁₀/PVA is constructed. In one embodiment, the surface coating composed of (PLGA/PLO)₁₅/PVA is constructed. In one embodiment, the surface coating composed of PLO(PLGA/PLO)₃/PVA is constructed. In one embodiment, the surface coating composed of PLO(PLGA/PLO)₅/PVA is constructed. In one embodiment, the surface coating composed of PLO(PLGA/PLO)₁₀/PVA is constructed. In one embodiment, the surface coating composed of PLO(PLGA/PLO)₁₅/PVA is constructed.

PLH/PLGA Multilayers

In some embodiments, the polyelectrolyte multilayers are PLH/PLGA multilayers that can be constructed by sequentially depositing PLH and PLGA on a surface of (i) PVA or PEG-coated polystyrene plate, (ii) PVA-crosslinked polystyrene plate, or (iii) PVA-crosslinked PTFE plate. Each depositing step comprises adding the PLH or PLGA solution to the plate surface, incubated for 10 min and washed 3 times for 2, 1, and 1 min.

In one embodiment, the surface coating composed of (PLGA/PLH)₃/PVA is constructed. In one embodiment, the surface coating composed of (PLGA/PLH)₅/PVA is constructed. In one embodiment, the surface coating composed of (PLGA/PLH)₁₀/PVA is constructed. In one embodiment, the surface coating composed of (PLGA/PLH)₁₅/PVA is constructed. In one embodiment, the surface coating composed of PLH(PLGA/PLH)₃/PVA is constructed. In one embodiment, the surface coating composed of PLH(PLGA/PLH)₅/PVA is constructed. In one embodiment, the surface coating composed of PLH(PLGA/PLH)₁₀/PVA is constructed. In one embodiment, the surface coating composed of PLH(PLGA/PLH)₁₅/PVA is constructed.

PLA/PLGA Multilayers

In some embodiments, the polyelectrolyte multilayers are PLA/PLGA multilayers that can be constructed by sequentially depositing PLA and PLGA on a surface of (i) PVA or PEG-coated polystyrene plate, (ii) PVA-crosslinked polystyrene plate, or (iii) PVA-crosslinked PTFE plate. Each depositing step comprises adding the PLA or PLGA solution to the plate surface, incubated for 10 min and washed 3 times for 2, 1, and 1 min.

In one embodiment, the surface coating composed of (PLGA/PLA)₃/PVA is constructed. In one embodiment, the surface coating composed of (PLGA/PLA)₅/PVA is constructed. In one embodiment, the surface coating composed of (PLGA/PLA)₁₀/PVA is constructed. In one embodiment, the surface coating composed of (PLGA/PLA)₁₅/PVA is constructed. In one embodiment, the surface coating composed of PLA(PLGA/PLA)₃/PVA is constructed. In one embodiment, the surface coating composed of PLA(PLGA/PLA)₅/PVA is constructed. In one embodiment, the surface coating composed of PLA(PLGA/PLA)₁₀/PVA is constructed. In one embodiment, the surface coating composed of PLA(PLGA/PLA)₁₅/PVA is constructed.

Quartz Crystal Microbalance-Dissipation (QCM-D) Measurement

QCM experiments were performed under Q-Sense E4 (Biolin Scientific AB/Q-sence, Sweden). The silicon oxide (SiO₂) coated quartz crystal chips (AT-cut quartz crystals, f0=5 MHz) were cleaned in 0.1M sodium dodecyl sulfate, followed by rinsing with Milli-Q water, drying under nitrogen, and exposing to oxygen plasma for 20 seconds. For QCM-D measurement, the chamber was stabilized to 25 degree C. and all measurements were recorded at the third overtone (15 MHz). To simulate the serial surface coating, the concentration and the washing conditions of each coating step in the QCM-D chamber are identical. About 1% bovine serum albumin (BSA, Millipore, Bedford, MA) was used for non-specific adsorption investigation and was introduced to chambers on the surface.

Surface Chemical Analysis

The chemical composition of the surface coating of the present disclosure was analyzed by X-ray photoelectron spectroscopy (XPS; VersaProbe III, PHI) with C₆₀ (10 kV, 10 nA) etching on silicon wafer. The pass energy used was 93.9 eV at steps of 0.5 eV. The relative atomic concentrations of carbon, nitrogen, oxygen and silicon were measured in the layer of samples to a maximum thickness of 10 nm.

Surface Roughness Measurement by Using Atomic-Force Microscope (AFM)

The roughness of the surface coating of the present disclosure was measured using atomic force microscope (AFM; Nanowizard 3, JPK instrument) with tapping mode. Silicon cantilevers with a resonant frequency of 134 kHz were utilized for the experiments.

### Example 2: Formation of Single-Cell Derived Spheroids

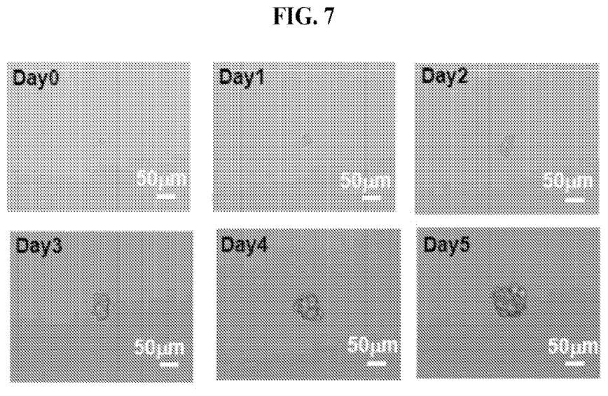

FIG. 7 shows the time-lapse microscope observation of HCT116 colorectal cancer cells cultured on the surface coating of the present disclosure on day 0, 1, 2, 3, 4 and 5 during the growth of the cancer cells supplied with complete DMEM medium. (Image photographed by Leica DMI6000B time-lapse microscope under 10× objective).

### Example 3: Generation of Cell Line-Derived Tumor Spheroids

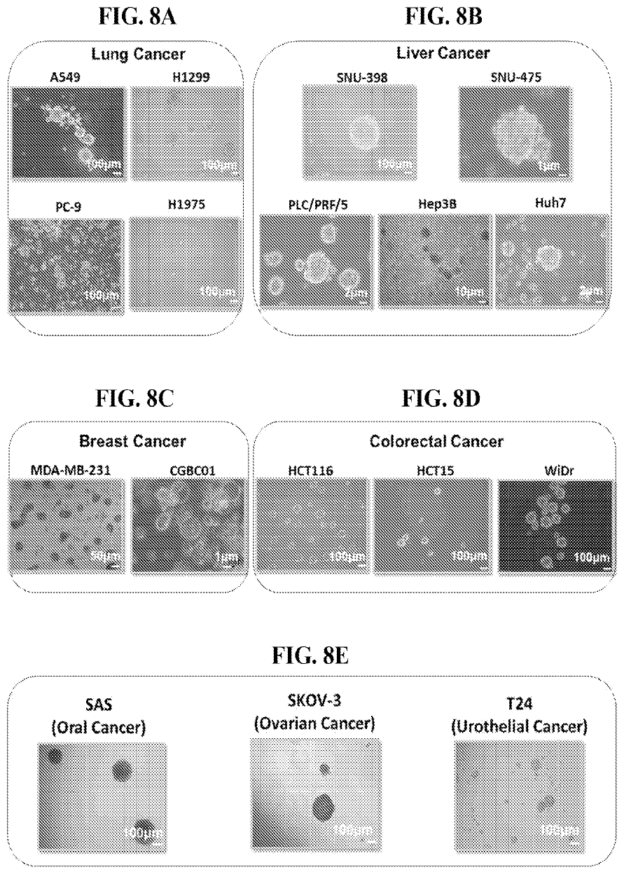

The surface coating of the present disclosure provides a biocompatible multilayer coated surface that enables cell adhesion for cell proliferation, and also provides non-fouling characteristic for spheroid formation directly on the surface. The cell culture system comprising the surface coating of the present disclosure was tested with various cancer cell lines and resulted in the successful cultivation and formation of spheroids derived from various cancer cell lines (shown in FIG. 8A-E). FIGS. 8A-E show the results of ex vivo cultivation using the culture platform of the invention, and the formation of spheroids (after 7-14 days) derived from (A) lung cancer cell lines A549, H1299, PC-9 and H1975; (B) liver cancer cell lines SNU-398, SNU-475, PLC/PRF/S, Hep3B and Huh7 (C) breast cancer cell lines MDA-MB-231 and CGBC01; (D) colorectal cancer cell lines HCT116, HCT15 and WiDr; and (E) human tongue squamous carcinoma cell line SAS, ovarian cancer cell line SK-OV-3, and cell line T24 derived from a human urinary bladder cancer patient. These cancer cells were grown on the culture system of the invention for 7 to 14 days (the number of seeding cells is about 1000).

### Example 4: Generation of CTC-Derived Tumor Spheroids

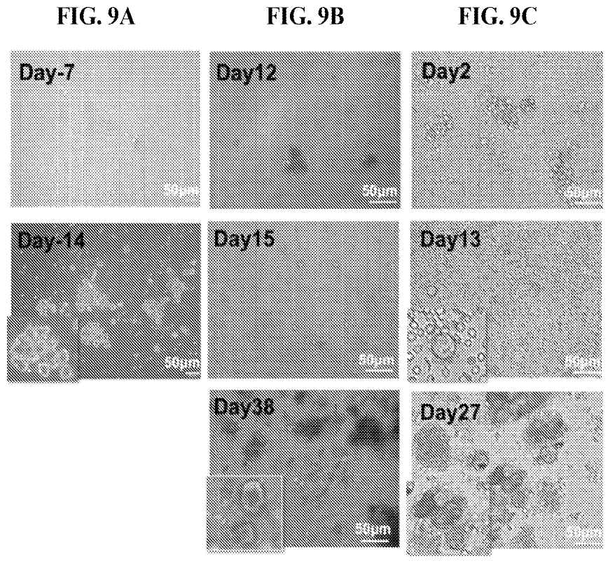

The cell culture system comprising the surface coating of the present disclosure was tested with various patient-derived CTCs and resulted in the successful cultivation and formation of spheroids derived from patient-derived CTCs (shown in FIG. 9A-C). FIGS. 9A-C show the representative time-dependent images of CTC-derived spheroid cultivation on the culture platform of the invention. (A) CTCs were isolated from a blood sample of a breast cancer patient; CTC-derived spheroids formed after 14 days. (B) CTCs were isolated from a blood sample of a head&neck cancer patient; CTC-derived spheroids formed after 38 days. (C) CTCs were isolated from a blood sample of a colorectal cancer patient; CTC-derived spheroids formed after 13-27 days. Scale bar: 50 μm.

The spheroids generated thereof may further benefit for future diagnosis and guidance in medical treatment and application, ex: non-invasive early cancer detection, personal medicine guidance, pre- and post-treatment drug resistance investigation, cell activity evaluation for immune cell-based cancer therapy, and provide substantial material to elucidate the mechanism participated in cancer progression by using the ex vivo cultivated patient-derived primary CTC cells.

### Example 5: Generation of Tissue-Derived Tumor Spheroids

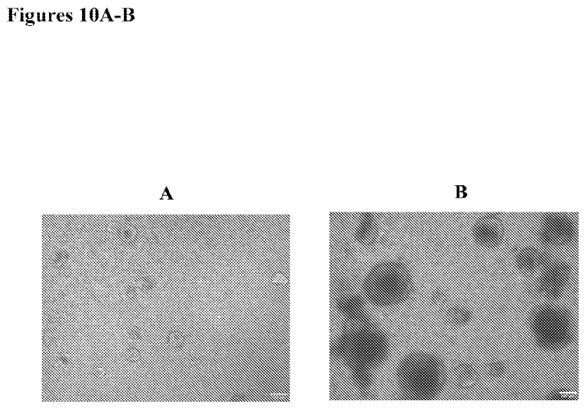

Primary tissue cells derived from animal model primary xenografts were cultured on the cell culture system of the invention for 7 days. FIGS. 10A-B show the images of tumor spheroids derived from primary colorectal tumor tissues obtained from colorectal cancer (CRC) patient. Tumor spheroids were generated on the culture platform of the invention after 2 weeks and 4 weeks. The results indicated that the cell culture platform of the invention is capable of forming tumoroids from primary tissue cells.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-31. (canceled)

32. A composition for coating a cell culture article, the composition comprising:
a) a polymer selected from the group consisting of poly(vinyl alcohol) (PVA), poly(ethylene glycol) (PEG), PEG-acrylate, polyvinylpyrrolidone (PVP), poly-L-lactide (PLLA), poly-D-lactide (PDLA), poly(L-lactide-co-D,L-lactide) (PLDLLA), poly(glycolic acid) (PGA), poly(lactic-co-glycolic acid) (PL-co-GA), poly(methyl methacrylate) (PMMA), poly(hydroxyethyl methacrylate) (p-HEMA) and derivatives thereof, wherein the polymer is crosslinked to a surface of the cell culture article, and
b) polyelectrolyte multilayers, wherein the polymer is in direct contact with a polycation or a polyanion of the polyelectrolyte multilayers.

33. The composition of claim 32, wherein the polymer is PVA, PEG, PEG-acrylate, PVP, PMMA or a derivative thereof.

34. The composition of claim 32, wherein the polymer is PVA, PEG or PEG-acrylate.

35. The composition of claim 32, wherein the polycation is a poly(amino acid) and/or the polyanion is a poly(amino acid).

36. The composition of claim 32, wherein the polycation is selected from the group consisting of poly(L-lysine) (PLL), poly(L-arginine) (PLA), poly(L-ornithine) (PLO), poly(L-histidine) (PLH), and a combination thereof.

37. The composition of claim 32, wherein the polyanion is poly(L-glutamic acid) (PLGA), poly(L-aspartic acid) (PLAA), or a combination thereof.

38. The composition of claim 32, wherein the polyelectrolyte multi-layers comprise at least one bilayer composed of a polycation and a polyanion (polycation/polyanion), wherein the bilayer is selected from the group consisting of PLL/PLGA, PLL/PLAA, PLA/PLGA, PLA/PLAA, PLO/PLGA, PLO/PLAA, PLH/PLGA, PLH/PLAA, and a combination thereof.

39. The composition of claim 32, wherein the polyelectrolyte multilayers are formed via layer-by-layer assembly.

40. The composition of claim 32, wherein the polyelectrolyte multi-layers comprise n bilayers, wherein n is an integer number ranging from 1 to 30, and wherein the outermost layer is a polycation or a polyanion.

41. The composition of claim 32, wherein the polyelectrolyte multilayers comprise n bilayers of (polycation/polyanion), and an additional layer of polyanion, wherein n is an integer number ranging from 1 to 30, and wherein the outermost layer is a polyanion.

42. The composition of claim 32, wherein the polyelectrolyte multilayers comprise n bilayers of (polyanion/polycation), and an additional layer of polycation, wherein n is an integer number ranging from 1 to 30, and wherein the outermost layer is a polycation.

43. A method for preparing the composition of claim 32, the method comprising the steps of:
a) providing a cell culture article having a hydrophobic surface,
b) modifying the hydrophobic surface with a treatment, wherein the treatment is a plasma treatment, corona discharge, UV ozone treatment or a hydrosilylation,
c) crosslinking a polymer to the modified surface, wherein the hydrophilic polymer is selected from the group consisting of PVA, PEG, PEG-acrylate, PVP, PLLA, PDLA, PLDLLA, PGA, PL-co-GA, PMMA and p-HEMA, and
d) sequentially depositing on the polymer alternating layers of polycations and polyanions to form the composition.

44. The method of claim 43, wherein the polymer is PVA, PEG or PEG-acrylate.

45. A cell culture system, comprising a cell culture article having a surface coated with the composition of claim 32.

46. The cell culture system of claim 45, further comprising cells and/or culture media.

47. A method for culturing cells, the method comprising:
a) providing a cell culture article having a surface coated with the composition of claim 32,
b) seeding cells on the coated surface, and
c) culturing the cells under a suitable medium for a sufficient period of time to form one or more spheroids.

48. The method of claim 47, wherein the one or more spheroids are generated via single cell proliferation.

49. The method of claim 47, wherein the one or more spheroids have an average diameter between 50 μM and 150 μM.

50. The method of claim 47, wherein the seeding comprises plating the cells at a density less than 1000 cells per cm² on the substrate.

51. A method for obtaining and optionally characterizing single-cell-derived spheroid, the method comprising:
a) culturing a heterogeneous population of cells on a surface coated with the composition of claim 1 to obtain at least one single-cell-derived spheroid disposed on the surface, and
b) optionally, analyzing the single-cell-derived spheroid to thereby obtain at least one characteristic of the single cell.
